(19) United States
(12) Patent Application Publication
(10) Pub. No.: US 2024/0366555 A1
(43) Pub. Date: Nov. 7, 2024

(54) SELECTIVE MTORC2 INHIBITOR AND USES THEREOF

(71) Applicant: ALIAD BIOPHARMA, Gyeonggi-do (KR)

(72) Inventors: Hyockman KWON, Seoul (KR); Yunhee KIM, Seoul (KR); Young Taek HAN, Cheonan-si (KR); Ki Soon SHIN, Seoul (KR)

(21) Appl. No.: 18/284,367

(22) PCT Filed: Apr. 11, 2022
(86) PCT No.: PCT/KR2022/005189

§ 371 Date: Sep. 27, 2023

(30) Foreign Application Priority Data
Apr. 13, 2021 (KR) 10-2021-0047686

(51) Int. Cl. A61K 31/352; A61P 25/22; A61P 25/28

(52) U.S. Cl. CPC A61K 31/352; A61P 25/22; A61P 25/28

(57) ABSTRACT

The composition comprising athyriol or a pharmaceutically acceptable salt thereof as an active ingredient, according to the present application, does not inhibit mTORC1 activity but selectively inhibits mTORC2 in an effective concentration and thus may be effectively used for preventing or treating mTORopathies, particularly autism spectrum disorder and neurodevelopmental disorders, which are caused by hyperactivated mTORC2.

### TECHNICAL FIELD

The present invention relates to a compound which selectively inhibits mTORC2, a preparation method thereof, and a composition comprising the same as an active ingredient.

### BACKGROUND ART

Autism or Autism Spectrum Disorder (ASD) is a neurodevelopmental disorder whose main symptoms are social communication disorder and repetitive behaviors, and the like. Although the prevalence rate is very high as 1.5% or more, there is no therapeutic agent for causes other than symptom relievers, so it costs huge social costs. According to U.S. statistics in 2011, it was investigated that the social costs of ASD reached 60 billion dollars per year, and costs of 40,000˜ 60,000 dollars per 1 child per year were spent on behavior correction in addition to medical expenses.

ASD is a very complex disease, and mutations are found in various genes having different functions in ASD patients. Only 8˜15% of ASD are associated with single gene mutation, but 50% or more of them correspond to mTORopathy directly affecting the PI3K/mTOR signaling pathway (Skelton et al. (2019) Mol. Neuropsychiatry 5:60-71). mTORopathy is a genetic disease in which the mTOR signaling pathway is overactivated by germline or somatic mutations in neurons, resulting in neurological abnormalities. Epilepsy, autism spectrum disorder (ASD), macrocephaly, tuberous sclerosis complex (TSC), seizure, fragile X syndrome (FXS), PTEN harmartoma tumor syndrome (PHTS), neurofibromatosis and intellectual disability and the like correspond to mTORopathy, and it is noteworthy that autism symptoms are common in patients with diseases corresponding to mTORopathy.

mTOR (mammalian target of rapamycin) is a serin/threonine protein kinase belonging to the phosphatidylinositol 3-kinase-related kinase (PIKK) family. In mammals, mTOR is a catalytic subunit shared by two different protein complexes, named mTOR complex 1 (mTORC1) and mTOR complex 2 (mTORC2), respectively. Two protein complexes act as a signal hub that regulates cell metabolism, growth, proliferation and survival by integrating intracellular and extracellular signals, and mTORC1 and mTORC2 have different characteristics in protein complex composition, substrate specificity and regulatory mechanism, so they are in charge of unique regions creating specific cellular responses depending on the type of signal transmitted from the upper level (Saxton & Sabatini (2017) Cell 168:960-976).

mTORC1 senses the level of available intracellular energy and nutrients to regulate anabolic actions to be progressed only under conditions where energy and nutrients are sufficient. For example, when the level of intracellular energy and nutrients is sufficiently high, mTORC1 phosphorylates 4E-BP1 (eukaryotic translation initiation factor 4E-binding protein 1) to activate cap-dependent protein synthesis, and in addition, activates SREBP (sterol responsive element binding protein) to facilitate de novo lipid synthesis. In addition, mTORC1 phosphorylates ULK1 and TFEB (transcription factor EB) to inhibit autophagy, thereby inhibiting the progress of catabolic actions. Therefore, as mTORC1 promotes anabolic actions and inhibits catabolic actions at the same time when the level of intracellular energy and nutrients is high, and conversely, it inhibits anabolic actions and promotes catabolic actions when the level of intracellular energy and nutrients is low, it maintains balance of anabolic and catabolic actions according to the level of intracellular energy and nutrients.

In contrast to the mTORC1 function, mTORC2 plays a role in inducing cells to survive or proliferate, or interact with each other at the appropriate place and time by receiving signals transmitted from outside the cells. For example, when the PI3K-dependent signaling pathway is stimulated by insulin or growth factors, mTORC2 is activated to promote survival and proliferation of cells. In addition, mTORC2 regulates cell shape change and migration and in particular, regulates synapse formation and function in neurons by organizing cytoskeleton through activation of Rho and Rac.

As described above, mTORC1 maintains equilibrium of anabolic actions and catabolic actions according to the level of intracellular energy and nutrients, and mTORC2 is a unique signal hub regulating cell survival, proliferation, and intracellular communication in response to extracellular signals. mTORC1 and mTORC2 have different characteristics each other in protein complex composition, substrate specificity, and regulatory mechanism. As such, considering that mTORC1 and mTORC2 perform separate functions, respectively, a method of inhibiting the corresponding mTORC signaling pathway, but not inhibiting the other mTORC signaling pathway for treating diseases in which one mTORC overactivation has a pathophysiological cause is preferable to minimize cytotoxicity and drug side effects.

Therefore, in order to develop a therapeutic agent for treating a disease caused by mTORC2 overactivation, development of a selective mTORC2 inhibitor is urgently needed. mTORC2 inhibitors developed so far are classified into three groups, but a selective mTORC2 inhibitor with a true meaning has not been developed yet.

First, rapamycin and its derivative, rapalog inhibit mTORC2. Although rapamycin and rapalog were known as mTORC1 selective inhibitors, it was found that long-term administration also inhibited mTORC2 activity (Sarbassov et al. (2006) Mol. Cell 22:159-168). Rapamycin binds to FKBP12 first, and then an FKBP12-rapamycin complex binds to an FKBP12-rapamycin-binding (FRB) domain of mTOR to inhibit the binding of mTORC1 substrate such as S6K1 and 4EBP1 to mTOR. However, in mTORC2, Rictor prevents the binding of the FKBP12-rapamycin complex to the FRB domain of mTOR, and accordingly, mTORC2 is not inhibited by rapamycin (Scaiola et al. (2010) Sci. Adv. 6: eabc1251). However, the FKBP12-rapamycin complex can bind to mTOR which does not form a complex, and long-term treatment inhibits de novo complex formation of mTORC1 and mTORC2. Consequently, during long-term treatment, rapamycin acts as a dual mTORC inhibitor which inhibits both mTORC1 and mTORC2 activities.

Second, an ATP-competitive mTOR kinase inhibitor which inhibits ATP binding at a catalytic site of mTOR competitively inhibits mTORC2. However, mTOR is a catalytic structural protein shared by mTORC1 and mTORC2, so the ATP-competitive mTOR kinase inhibitor inevitably inhibits mTORC1 and mTORC2 activities.

Third, a protein-protein interaction modulator preventing mTORC2 complex formation has been developed as a third-generation mTORC2 inhibitor. However, mTORC2 has a very large size over a mega Dalton, and consists of at least 6 structural proteins, so it is very difficult to develop an effective and selective small molecule mTORC2 inhibitor. So far, only one kind of compound inhibiting binding of Rictor and mTOR has been reported as an mTORC2 protein-protein interaction modulator, but its use is limited due to its low inhibitory activity (Benavides-Serrato et al. (2017) PLOS ONE 12: e0176599).

### DISCLOSURE

### Technical Problem

One object of the present invention is to provide a pharmaceutical composition for prevention or treatment of mTORopathy, comprising athyriol or a pharmaceutically acceptable salt thereof as an active ingredient.

Another object of the present invention is to provide a food composition for prevention or improvement of mTORopathy, comprising athyriol or a sitologically acceptable salt thereof as an active ingredient.

Other object of the present invention is to provide a food composition for enhancing memory, comprising athyriol or a sitologically acceptable salt thereof as an active ingredient.

Other object of the present invention is to provide a method for prevention or treatment of mTORopathy comprising administering a therapeutically effective amount of a composition comprising athyriol or a pharmaceutically acceptable salt thereof into a subject in need thereof.

Other object of the present invention is to provide a use of a composition comprising athyriol or a pharmaceutically acceptable salt thereof for preparing a drug for prevention or treatment of mTORopathy.

### Technical Solution

The present inventors have made intensive efforts to find a compound which can exhibit a therapeutic effect on autism spectrum disorder (ASD) among mTORopathy, which is an mTOR (mammalian target of rapamycin) pathway-related disease, and as a result, they have found that a small molecule compound derived from a natural substance, athyriol recovers behavioral and neurophysiological disorders associated with autism spectrum disorder in Pten KO mice, which are autism spectrum disorder model animals, and athyriol and its derivative, norathyriol inhibits mTORC2 in an endosomal location-selective manner and inhibits mGluR-dependent LTD formation evaluated as a fundamental cause of autistic symptoms, thereby completing the present invention.

Hereinafter, the present invention will be described in more detail.

In the composition and method provided in the present description, unless otherwise mentioned, as an effective (active) ingredient, not only athyriol, but also a pharmaceutically acceptable salt, hydrate, solvate, isomer (for example, optical isomer), and/or derivative thereof may be used, and all of these should be construed as being included within the scope of the present invention.

The present invention provides a pharmaceutical composition for prevention or treatment of mTORopathy comprising athyriol (1,6,7-Trihydroxy-3-methoxy-9H-xanthen-9-on) represented by the following Chemical formula 1 or a pharmaceutically acceptable salt as an active ingredient:

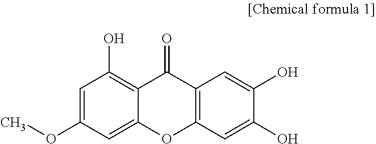 [SMILES: COc1cc(O)c2c(=O)c3cc(O)c(O)cc3oc2c1]

In one example of the present invention, the athyriol represented by Chemical formula 1 may be synthesized by a sequential alkylation reaction from norathyriol (1,3,6,7-tetrahydroxy-9H-xanthen-9-one) represented by the following Chemical formula 2:

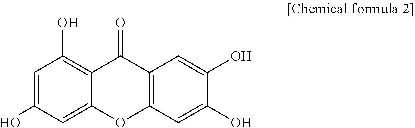 [SMILES: O=c1c2cc(O)c(O)cc2oc2cc(O)cc(O)c12]

In the present invention, the “mTORopathy” refers to an mTOR (mammalian target of rapamycin) pathway-related disease. Specifically, “mTORopathy” is a concept including neurological diseases in which the mTOR signaling pathway is overactivated by germline or somatic mutations in neurons and neurological abnormalities are exhibited, and it may be at least one selected from the group consisting of epilepsy (Moloney et al. (2021) Brain Comm. 3:1-21), autism spectrum disorder (ASD) (Winden et al. (2018) Ann. Rev. Neurosci. 41:1-23), macrocephaly (Butler et al., (2005) J. Med. Genet. 42:318-321), tuberous sclerosis complex (TSC) (Crino (2015) Cold Spring Harb. Perspect. Med. 5: a022442), seizure (Harvey et al. (2008) Epilepsia 49:146-155), fragile X syndrome (Sharma et al. (2010) J. Neurosci. 30:694-702), PTEN harmartoma tumor syndrome (PHTS) (Endersby & Baker (2008) Oncogene 27:5416-5430), neurofibromatosis (Johannessen et al. (2005) Proc. Natl. Acad. Sci. USA 102:8573-8578) or intellectual disability (Dentel et al. (2019) Neuron 104:1032-1033).

In the present invention, the “autism spectrum disorder” is a concept including neurodevelopmental disorders characterized by communication, social interaction and flexibility disorder of thought and action, and it may be at least one selected from the group consisting of autism, Asperger's disorder, Pervasive Development Disorder-Not Otherwise Specified (PDD-NOS), Rett's disorder, Childhood Disintegrative Disorder and Autism Spectrum Disorder.

In the present invention, the “Autism Spectrum Disorder” preferably includes at least one symptom selected from the group consisting of hyperactivity symptoms, symptoms of lack of sociability, and epileptiform convulsion symptoms, but not limited thereto, and any one may be included as long as it is a symptom reported as a symptom of Autism Spectrum Disorder.

In the present invention, “Tuberous Sclerosis Complex” is an autosomal genetic disease of which cause is a loss-of-function mutation in TSC1 or TSC2. TSC is a regulator which inhibits mTORC1 activity, and in tuberous sclerosis complex patients, the mTOR signaling pathway is overactivated.

In the present invention, “PTEN harmartoma tumor syndrome (PHTS)” is an autosomal genetic disease of which cause is a loss-of-function mutation in PTEN. PTEN is a regulator which inhibits the AKT/mTOR pathway, and in PHTS patients, the mTOR signaling pathway is overactivated. PTEN mutation in which a lipid phosphatase domain is maintained has been found as a cause of sporadic autism accompanied by macrocephaly.

In the present invention, “fragile X syndrome” is a genetic disease of which cause is lack of FMRP1 (fragile X mental retardation protein 1). FMRP1 is a translational repressor inhibiting protein translation of hundreds of mRNAs in a brain. In Fmr1-/y mutant mice, the mTOR signaling pathway is overactivated, and protein synthesis increases, and mGluR-dependent LTD excessively occurs. In 30% or more of FXS patients, autism spectrum disorder occurs.

Since autism spectrum disorder symptoms are likely to be accompanied in the tuberous sclerosis complex, PTEN harmartoma tumor syndrome and fragile X syndrome occurring mutation in the mTOR signaling pathway, the mTOR signaling pathway is known as an etiological hub of autism spectrum disorder.

In the present invention, “epilepsy” is one of chronic nerve disorders which may occur by tuberous sclerosis caused by excessive activation of mTOR, and refers to a disease which causes seizure and convulsion by generating electricity in a brain due to irregular stimulation of the brain nerve cells. In epilepsy patients, TSC, PI3K, and AKT mutations regulating the mTOR signaling pathway are found, and epilepsy occurs in TSC KO mice and PTEN KO mice.

In the present invention, “neurofibromatosis” is an autosomal genetic disease of which cause is a loss-of-function mutation in NF1. NF1 is a GTPase-activating protein inhibiting Ras which is a proto-oncogene, and in neurofibromatosis patients, intellectual disorder, attention deficit, hyperactivity disorder, sleep disorder, anxiety disorder and the like are accompanied with benign or malignant tumor occurrence in the brain, skin, bone, kidney and the like. In Nf1+/− mice, the PI3K/mTOR signaling pathway is overactivated, and it has been found that overactivation of the PI3K/mTOR signaling pathway is a cause occurring tumor in neurofibromatosis.

The composition may further comprise any one or more selected from the group consisting of norathyriol, mangiferin, neomangiferin, and pharmaceutically acceptable salts thereof.

In addition, the present invention provides a composition for inhibiting mTORC2 activity comprising any one or more selected from the group consisting of athyriol, norathyriol, mangiferin, neomangiferin), and pharmaceutically acceptable salts thereof.

The composition for inhibiting mTORC2 activity may be a pharmaceutical composition or food composition, but not limited thereto.

The prevention or treatment effect of mTORopathy, particularly, autism spectrum disorder of the pharmaceutical composition of the present invention will be described later in the following examples.

When a loss-of-function mutation occurs in a PTEN (phosphatase and tensin homolog) gene acting as a negative regulator in the PI3K/mTOR signaling pathway, the mTOR signaling pathway is overactivated. It has been known that autism symptoms such as macrocephaly, excessive dendritic arborization, mental retardation, and the like appear in PTEN mutant patients, and PTEN mutation is found in 1%˜5% of ASD patients. In addition, when loss-of-function mutation occurs in TSC1/2, the mTOR signaling pathway is overactivated, and in 89% of TSC patients, abnormal structures such as cerebral cortical nodules and the like are formed and macrocephaly occurs, and autism spectrum disorder symptoms such as reduced neuroplasticity appear (Vignoli et al. (2015) Orphanet J. Rare Dis. 10:154). FMRP (Fragile X mental retardation protein 1) is an RNA binding protein, and a single causative gene of FXS inhibiting a translation process by binding to 400 or more mRNAs in a brain. A decrease in FMRP by a CGG repeat sequence is accompanied by autism spectrum disorder symptoms such as cognitive function and sociability decline, epileptic seizure, macrocephaly and the like. In 5% of ASD patients, FMRP mutation is found. FMRP knockout (KO) mice have high mTOR activity and increased protein synthesis by 20%. Duplication of 15q11-13 regions is observed in about 1% of ASD patients, and CYFIP1 (cytoplasmic FMR 1 interacting protein 1) among the genes located in the 15q11-13 region is excessively expressed in brains of patients. CYFIP1 regulates microfilament formation and binds to FMRP to inhibit mRNA translation initiation. It has been known that excessive arborization of dendritic spines and excessive activation of the mTOR pathway are observed in CYFIP1 overexpressing transgenic mice. As such, the PI3K/mTOR signaling pathway is located in an etiological hub of ASD.

Despite the complexity of ASD in the symptoms as described above, two neurophysiological abnormalities commonly occur in ASD patients, and one of them is an excessive dendritic arborization phenomenon and the other is occurrence of abnormal long-term depression (LTD) (Piochon et al. (2016) Nat. Neurosci. 19:1299-1310). The excessive dendritic arborization and abnormal LTD formation provide major causes of cognitive and behavioral deficits shown in ASD. A remarkable aspect is that overactivation of the mTOR signaling pathway, particularly, overactivation of mTORC2 is very likely to mediate excessive dendritic arborization and abnormal LTD formation. LTD is a phenomenon in which transmission efficiency of excitatory synapses decreases and is weakened over a long period of time after applying a suitable form of stimulation for a long time. In the hippocampus, it is found in the synapses between Schaffer collateral from CA3 and CA1 pyramidal cells. mGluR (metabotropic glutamate receptor)-dependent LTD among LTDs are not spread to other surrounding dendrites, and are formed as new proteins are rapidly synthesized in dendrites. Representatively, Arc (activity-regulated cytoskeleton-associated protein) mRNA begins to be translated when mGluR is activated in dendritic spines in which LTD is formed, and protein is synthesized, and Arc forms LTD by binding to endophilin-3 and dynamin2 to promote endocytosis. This mGluR-dependent LTD contributes to the plasticity of neural networks that occur in the process of registering and modifying new experiences in the hippocampus, and the mGluR-dependent LTD abnormality in CA1 neurons provides a major cause for cognitive and behavioral deficits in ASD. In fact, mGluR-dependent LTD appears very large in FXS, and SHANK3 and Ube3a mediating mGluR signals have been found as ASD risk factors.

Excessive dendritic arborization, one of common neurophysiological abnormalities found in ASD patients is caused by lacking synaptic pruning during the postnatal neurodevelopmental process establishing neural circuits, and when abnormality occurs in LTD formation, synaptic pruning is reduced. Therefore, mGluR-dependent LTD abnormality may be a major cause of ASD occurrence, and pharmacological adjustment of dysregulation generated during the LTD formation process can provide a treatment method for ASD symptoms. It is known that mTORC2 acts as an essential factor for mGluR-dependent LTD formation (Zhu et al. (2018) Nature Neurosci. 21:799-802). In mTORC2-deficient animals, mGluR-dependent LTD was not formed, and behavioral ability associated with mGluR-dependent LTD was also lacking. On the other hand, no change in mGluR-dependent LTD was observed in mTORC1-deficient animals. In addition, mTORC2 is likely involved in dendritic spine formation and morphological changes. Cofilin is a factor inducing disassembly of actin filaments and acts as a major regulator determining activity-dependent synapse plasticity and the form of dendritic spine. Since cofilin signaling is regulated by mTORC2, if mTORC2 was excessively activated, excessive arborization of dendrites and morphological changes in dendritic spines observed in ASD could be induced.

The athyriol or pharmaceutically acceptable salt thereof and norathyriol or pharmaceutically acceptable salt of the present invention can selectively inhibit mTORC2 (mammalian target of rapamycin complex 2) activity. Specifically, the athyriol or pharmaceutically acceptable salt thereof and norathyriol or pharmaceutically acceptable salt thereof can inhibit mTORC2 activity twice or more than mTORC1 activity.

The athyriol or pharmaceutically acceptable salt thereof and norathyriol or pharmaceutically acceptable salt of the present invention can inhibit mTORC2 activity at an effective concentration in in vitro mTOR kinase assay, whereas it cannot inhibit mTORC1 activity.

The athyriol or pharmaceutically acceptable salt thereof and norathyriol or pharmaceutically acceptable salt of the present invention can selectively inhibit mTORC2 (mammalian target of rapamycin complex 2) activity in an endosome-location-selective manner.

The athyriol or pharmaceutically acceptable salt thereof and norathyriol or pharmaceutically acceptable salt of the present invention can inhibit mTORC2 activity located in endosome at an effective concentration in LocaTOR2 assay, whereas it cannot inhibit mTORC2 activity located in a cell membrane.

In the present invention, “selective mTORC2 inhibition” means an agent with relatively high efficacy inhibiting mTORC2 activity compared to efficacy inhibiting mTORC1 activity in in vitro mTOR kinase assay, and refers to a case in which IC_{50, mTORC1} is twice or higher than IC_{50, mTORC2}.

In the present invention, “endosome-location-selective mTORC2 inhibition” means an agent with relatively high efficacy inhibiting mTORC2 activity located in endosome compared to efficacy inhibiting mTORC2 activity located in a cell membrane (plasma membrane) in LocaTOR2 assay, and refers to a case in which IC_{50, PM} is twice or higher than IC_{50, endosome}.

The athyriol or pharmaceutically acceptable salt thereof and norathyriol or pharmaceutically acceptable salt of the present invention can inhibit mGluR-dependent mTORC2 activation and Arc expression in synapses of primary cultured neurons.

The athyriol or pharmaceutically acceptable salt thereof and norathyriol or pharmaceutically acceptable salt of the present invention can inhibit the increases in mGluR-dependent mTORC2 activity and Arc expression induced by 3,5-dihydroxyphenylglycine (DHPG), which is an mGluR1/5 agonist, in synapses of primary cultured neurons.

The athyriol or pharmaceutically acceptable salt thereof and norathyriol or pharmaceutically acceptable salt of the present invention can inhibit mGluR-dependent LTD formation in hippocampal brain slices.

The athyriol or pharmaceutically acceptable salt thereof and norathyriol or pharmaceutically acceptable salt of the present invention can inhibit mGluR-dependent LTD occurrence induced by DHPG in hippocampal brain slices.

The athyriol or pharmaceutically acceptable salt thereof and norathyriol or pharmaceutically acceptable salt of the present invention can enhance memory ability and sociability of normal mice.

The athyriol or pharmaceutically acceptable salt thereof and norathyriol or pharmaceutically acceptable salt of the present invention can improve memory ability measured by a Y-maze test in normal mice, and can enhance sociability measured by a 3-chamber test.

The composition of the present invention may be administered as various oral and parenteral formulations during clinical administration, and when formulated, it is prepared using a diluent or excipient such as a commonly used filler, thickener, binder, wetting agent, disintegrating agent, surfactant, or the like.

Solid preparations for oral administration include tablets, powders, granules, capsules, troches, and the like, and these solid preparations are prepared by mixing at least one or more excipients, for example, starch, calcium carbonate, sucrose, lactose, or gelatin, or the like to at least one active substance of the present invention. In addition, lubricants such as magnesium stearate talc are used in addition to simple excipients. Suspensions, oral liquids, emulsions or syrups or the like corresponds to liquid preparations for oral administration, and in addition to water and liquid paraffin which are commonly used simple diluents, various excipients, for example, wetting agents, sweeteners, flavoring agents, preservatives, and the like may be comprised.

Preparations for parenteral administration include sterilized aqueous solutions, non-aqueous solvents, suspension solvents, emulsions, freeze-dried preparations, suppositories, and the like. As the non-aqueous solvent and suspension solvent, propylene glycol, polyethylene glycol, vegetable oil such as olive oil, injectable esters such as ethyl oleate, and the like may be used. As a base compound of the suppositories, witepsol, macrogol, tween 61, cacao butter, laurin butter, glycerol, gelatin, and the like may be used.

In addition, an effective dose for the human body of the composition of the present invention may vary depending on a patient's age, body weight, gender, administration form, health condition and degree of disease, and in general, it may be about 0.001-100 mg/kg/day, and preferably, 0.01-35 mg/kg/day. Based on an adult patient with a body weight of 70 kg, it is generally 0.07-7000 mg/day, preferably, 0.7-2500 mg/day, and it may be administered once to several times in divided doses a day at regular intervals depending on judgment of a doctor or pharmacist.

The active substance of the present invention may be used in a form of pharmaceutically acceptable salt, and as a salt, an acid addition salt formed by a pharmaceutically acceptable free acid is useful. Expression of the pharmaceutically acceptable salt is a concentration that has an effective action which is relatively non-toxic and harmless to patients, and means any organic or inorganic addition salt of a base compound of an active substance in which side effects caused by this salt do not diminish beneficial effects of the base compound of the active substance. These salts may use an inorganic acid and an organic acid as a free acid, and as the inorganic acid, hydrochloric acid, hydrobromic acid, nitric acid, sulfuric acid, perchloric acid, phosphoric acid, and the like may be used, and as the organic acid, citric acid, acetic acid, lactic acid, maleic acid, fumaric acid, gluconic acid, methanesulfonic acid, glycolic acid, succinic acid, tartaric acid, galacturonic acid, embonic acid, glutamic acid, aspartic acid, oxalic acid, (D) or (L) malic acid, methanesulfonic acid, ethanesulfonic acid, 4-toluenesulfonic acid, salicylic acid, citric acid, benzoic acid or malonic acid and the like may be used. In addition, these salts include alkali metal salts (sodium salts, potassium salts, etc.) and alkali earth metal salts (calcium salts, magnesium salts, etc.) and the like. For example, the acid addition salt may include acetate, aspartate, benzate, besilate, bicarbonate/carbonate, bisulfate/sulfate, borate, camsylate, citrate, edisylate, ethylate, formate, fumarate, glyceptate, gluconate, glucuronate, hexafluorophosphate, hybenzate, hydrochloride/chloride, hydrobromide/bromide, hydroiodide/iodide, isethionate, lactate, malate, maleate, malonate, methylate, methyl sulfate, naphthylate, 2-naphthylate, nicotinate, nitrate, orotate, oxalate, palmitate, pamoate, phosphate/hydrogen phosphate/dihydrogen phosphate, saccharate, stearate, succinate, tartrate, tosylate, trifluoroacetate, aluminum, alginine, benzathine, calcium, choline, diethyl amine, diolamine, glycine, lysine, magnesium, meglumine, olamine, potassium, sodium, tromethamine, zinc salt, and the like, and among them, hydrochloride or trifluoroacetate is preferable.

The acid addition salt according to the present invention may be prepared by filtering and drying precipitates produced by dissolving an active substance in an organic solvent, for example, methanol, ethanol, acetone, methylene chloride, acetonitrile, and the like, and adding an organic acid or inorganic acid by a common method, or may be prepared by distilling under reduced pressure and then drying a solvent and an excessive amount of acids or crystallizing them under an organic solvent.

Furthermore, a pharmaceutically acceptable metal salt can be made using a base. An alkali metal or alkali earth metal salt is obtained for example, by dissolving a compound in an excessive amount of alkali metal hydroxide or alkali earth metal hydroxide solution, filtering an undissolved compound salt, and evaporating and drying filtrates. Then, as a metal salt, preparing a sodium, potassium or calcium salt is pharmaceutically suitable. In addition, a silver salt corresponding thereto is obtained by reacting an alkali metal or alkali earth metal salt with a suitable salt (e.g., silver nitrate).

In addition, the present invention provides a food composition for prevention or improvement of mTORopathy, comprising athyriol or a sitologically acceptable salt thereof as an active ingredient.

Description for the athyriol and mTORopathy is as described above.

The composition may further comprise any one or more selected from the group consisting of norathyriol, mangiferin, neomangiferin, and sitologically acceptable salts thereof.

The food composition according to the present invention may be characterized in that it is a composition for food or food additives, but not limited thereto, and it may be usefully used as a food, for example, main raw material, supplementary raw material, food additive, health functional food, or functional beverage, effective in prevention or improvement of mTORopathy.

The food means a natural product or processed product containing one or more nutrients, and preferably, means what becomes in a state in which it can be directly eaten through a certain processing process, and as a common meaning, it refers to that including all foods, food additives, health functional foods and functional beverages.

Foods to which the food composition according to the present invention can be added include, for example, various kinds of foods, beverages, gum, tea, vitamin complexes, functional foods, and the like. In addition, in the present invention, the food includes special nutrient food (e.g., formula milk, infant, baby food, etc.), processed meat products, fish meat products, tofu, jelly, noodles (e.g., ramen, noodles, etc.), bread, health supplement foods, seasoning foods (e.g., soy sauce, soybean paste, red pepper paste, mixed paste, etc.), sauces, confectionery (e.g., snacks), candies, chocolate, gum, ice creams, dairy products (e.g., fermented oil, cheese, etc.), other processed foods, Kimchi, salted foods (various kinds of Kimchi, pickled vegetables, etc.), beverages (e.g., fruit juice beverages, vegetable beverages, soybean milk, fermented beverages, etc.), natural seasonings (e.g., ramen powder, etc.), but not limited thereto. The foods, beverages or food additives may be prepared by a common preparation method.

The health functional food means a processed food designed to sufficiently express body regulatory functions related to bio-defensive rhythm control, disease prevention and recovery and the like of a food group or food composition in which added value is given so that the function of the corresponding food can be applied and expressed for a specific purpose by using physical, biochemical, bioengineering methods and the like to food. In the functional food, a sitologically acceptable food supplement additive may be comprised, and a carrier, an excipient, and a diluent appropriate commonly used in preparation of the functional food may be further comprised.

In the present invention, the functional beverage refers to a general term for drinking to quench thirst or enjoy the taste, and there is no particular limitation on other components other than comprising the composition for improvement or prevention of mTORopathy symptoms as an essential component at the indicated ratio, and various flavors or natural carbohydrates or the like may be contained as an additional ingredient as a common beverage.

Furthermore, in addition to the aforementioned, the food containing the food composition for improvement or prevention of mTORopathy symptoms of the present invention may contain various nutrients, vitamins, minerals (electrolytes), flavors such as synthetic flavors and natural flavors and the like, coloring agents and fillers (cheese, chocolate, etc.), pectic acid and its salts, alginic acid and its salts, organic acids, protective colloidal thickeners, pH adjusting agents, stabilizers, preservatives, glycerin, alcohol, carbonating agents used in carbonated beverages, and the like, and the components may be used independently or in combination.

In a food containing the food composition of the present invention, the amount of the composition according to the present invention may be comprised as 0.001% by weight to 100% by weight of the total food weight, and preferably, it may be comprised as 1% by weight to 99% by weight, and in case of beverages, it may be comprised at a ratio of 0.001 g to 10 g, preferably, 0.01 g to 1 g on the basis of 100 ml, but in case of long-term intake for the purpose of health and hygiene or purpose of health regulation, it may be less than or equal to the above range, and the active ingredient can be used in an amount greater than or equal to the above range, since there is no problem in terms of safety, and therefore, it is not limited to the above range.

The food composition of the present invention may further comprise at least one excipient and/or freeze-drying agent.

In addition, the present invention provides a food composition for enhancement of brain or cognitive functions, comprising athyriol or a sitologically acceptable salt thereof as an active ingredient.

In the present invention, the “brain or cognitive functions” may be learning ability, memory or concentration, but not limited thereto.

Furthermore, the present invention provides a food composition for improving anxiety disorder, comprising athyriol or a sitologically acceptable salt thereof as an active ingredient.

In one embodiment of the present invention, as a result of treating athyriol into an autism spectrum disorder animal model and a normal animal model, it has been confirmed that athyriol exhibits a memory ability improvement effect and an anxiety disorder improvement effect, and therefore, the athyriol or a pharmaceutically acceptable salt thereof, and norathyriol or a sitologically acceptable salt thereof of the present invention can be usefully used as a use for memory enhancement or a use for anxiety disorder improvement.

Moreover, the present invention provides a method for prevention or treatment of mTORopathy comprising administering a therapeutically effective dose of a composition comprising athyriol or a pharmaceutically acceptable salt thereof into a subject in need thereof.

In the present invention, the term “therapeutically effective dose (or, effective amount)” means an amount very sufficient to deliver a desired effect, but appropriate enough to sufficiently prevent severe side effects within the medical judgment scope. The amount of the composition administered to the body by the composition of the present invention may be appropriately adjusted in consideration of the administration route and administration subject.

The “administration” means providing the prescribed pharmaceutical composition of the present invention into a subject by any appropriate method. Then, the subject refers to an animal, and may typically be a mammal which can exhibit a beneficial effect by treatment using the composition of the present invention. As a preferable example of this subject, a primate such as a human may be included. In addition, such subjects may include all subjects who have symptoms of an allergic disease or are at risk of having such symptoms.

In addition, the present invention provides a use for preventing or treating mTORopathy of a composition comprising athyriol or a pharmaceutically acceptable salt thereof.

Description of the athyriol and mTORopathy is as described above.

Furthermore, the present invention provides a use of a composition comprising athyriol or a pharmaceutically acceptable salt thereof for preparing a drug for prevention or treatment of mTORopathy.

In addition, other aspect of the present invention provides a preparation method of athyriol represented by Chemical formula A comprising synthesizing a compound represented by the following Chemical formula 2 by reacting norathyriol represented by the following Chemical formula 1 and Ph₂CCl₂ under presence of Ph₂O as shown in the following Reaction formula 1 (step 1);
synthesizing a compound represented by the following Chemical formula 3 by reacting the compound represented by Chemical formula 2 and Mel under presence of an organic solvent and a base catalyst (step 2); and
reacting a compound represented by Chemical formula 3 and CSA under an organic solvent (step 3).

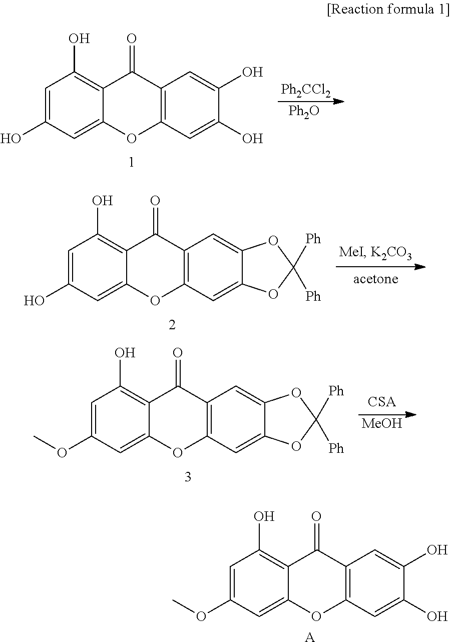

The present inventors have investigated that athyriol obtained by the above method has effects of inhibiting mTORC2 in an endosome-location-selective manner, inhibiting mGluR-dependent mTORC2 activation and Arc expression in synapses of primary cultured neurons, inhibiting mGluR-dependent LTD formation in hippocampal brain slices, alleviating excessive dendritic arborization by Cyfip1 overexpression in primary cultured neurons, and recovering behavioral and neurophysiological disorders related to autism in autism spectrum disorder model, Pten KO mice, so it is suitable for being used as a composition for prevention or treatment of autism spectrum disorder (ASD) and neurological diseases comprising mTORopathy of which cause is overactivation of the mTOR pathway.

### Advantageous Effects

The composition comprising athyriol or a pharmaceutically acceptable salt thereof according to the present application as an active ingredient does not inhibit mTORC1 activity at an effective concentration and selectively inhibits mTORC2, and therefore, it can be effectively used for prevention or treatment of mTORopathy, in which pathology is caused by overactive mTORC2, particularly, autism spectrum disorder and neurodevelopmental diseases.

### BRIEF DESCRIPTION OF THE DRAWINGS

FIG. 1 is diagrams of measuring that cognitive function is improved after administering athyriol into autism model Pten KO mice by a Y-type maze test and a passive avoidance test. FIG. 1a is a diagram of the Y-type maze test result, and FIG. 1b is a diagram of the passive avoidance test result.

FIG. 2 is diagrams of measuring that anxiety disorder is improved after administering athyriol into autism model Pten KO mice by an elevated plus maze test.

FIG. 3 is diagrams of measuring recovery of sociability after administering athyriol into autism model Pten KO mice by a social-open field test.

FIG. 4 is diagrams of measuring recovery of sociability after administering athyriol into autism model Pten KO mice by a 3-chamber test.

FIG. 5 is diagrams showing that macrocephaly symptoms are improved after administering athyriol into autism model Pten KO mice.

FIG. 6 are in vitro kinase assay analysis results showing that athyriol and norathyriol compounds of the present invention are selective inhibitors of mTORC2. FIG. 6a is the in vitro mTORC2 kinase assay analysis result showing that norathyriol inhibits mTORC2 activity, and FIG. 6b is the in vitro mTORC2 kinase assay analysis result showing that the mTORC2 inhibitory activity of athyriol is excellent compared to norathyriol. FIG. 6c is the in vitro mTORC1 kinase assay analysis result showing that norathyriol does not inhibit mTORC1 activity.

FIG. 7 is the immunoblot analysis result showing that the norathyriol compound inhibits a mTORC2 signaling pathway in cells. FIG. 7a is the immunoblot analysis result showing that norathyriol inhibits phosphorylation of Akt Ser473, which is an mTORC2 activity indicator, but does not inhibit phosphorylation of Akt Thr308, which is a PDK1 activity indicator. FIG. 7b is the immunoblot analysis result showing that norathyriol stabilizes NDRG1 protein and reduces the NDRG1 Thr346 phosphorylation ratio compared to NDRG1 protein at the same time. FIG. 7c is the immunofluorescent staining result showing that Fox03a moves from cytoplasm into a nucleus by norathyriol. FIG. 7d is the luciferase reporter assay analysis result showing that expression of the FHRE-synthetic luciferase gene increases by norathyriol.

FIG. 8 is drawings of measuring that norathyriol inhibits de novo mTORC2 complex formation by simultaneous immunoprecipitation analysis.

FIG. 9 is drawings of measuring that norathyriol inhibits mTORC2 located in endosome, but does not inhibit mTORC2 located in cell membranes by LocaTOR2 assay. FIG. 9a is the result of confirming the inhibition effect on mTORC2 located in cell membranes of A549 cells, in which KRas4B^{C30}-FKBP and FRB-AKT2 are introduced, by norathyriol. FIG. 9b is the result of confirming the inhibition effect on mTORC2 located in early endosome of A549 cells, in which Rab5-FKBP and FRB-AKT2 are introduced, by norathyriol. FIG. 9c is the result of confirming the inhibition effect on mTORC2 located in late endosome of A549 cells, in which Rab7-FKBP and FRB-AKT2 are introduced, by norathyriol. FIG. 9d is the result of confirming the inhibition effect on mTORC2 located in recycling endosome of A549 cells, in which Rab11-FKBP and FRB-AKT2 are introduced, by norathyriol. FIG. 9e is the result of confirming the inhibition effect on mTORC2 by norathyriol in mitochondria of A549 cells in which Bcl-XL-FKBP is introduced as an FKBP-recruiter, and FIG. 9f is that in ER of A549 cells in which TcRb-FKBP is introduced as an FKBP-recruiter. FIG. 9g is a diagram showing the results of quantifying FIG. 9a to FIG. 9f.

FIG. 10 is the result of immunofluorescent staining which shows that norathyriol inhibits excessive dendritic arborization induced by CYFIP1 overexpression in primary cultured hippocampal neurons. FIG. 10a is diagrams showing the confocal laser microscope scan result showing that norathyriol inhibits excessive dendritic arborization induced by CYFIP1 overexpression in primary cultured hippocampal neurons. FIG. 10b is the result of quantifying and showing the number of dendritic spines per unit neurite length, the number of dendritic spines per neurite, the number of dendritic spines per cell, and the number of dendritic spines in primary branching neurites according to norathyriol treatment.

FIG. 11 are diagrams showing the inhibitory efficacy of norathyriol on mGluR-dependent mTORC2 activation in synapses of cerebral neurons. FIG. 11a is the result of LocaTOR2 assay-immunofluorescent staining which shows that mTORC2 located in late endosome of dendritic spines is rapidly activated by DHPG, which is an mGluR5 agonist. FIG. 11b is the result of LocaTOR2 assay-immunofluorescent staining which shows that mTORC2 activation in late endosome induced by DHPG in dendritic spines is inhibited by norathyriol.

FIG. 12 are diagrams showing the inhibitory efficacy of norathyriol on mGluR-dependent Arc protein expression in synapses of cerebral neurons. FIG. 12a is the result of immunofluorescent staining which shows that Arc protein synthesis rapidly induced by DHPG is inhibited by norathyriol. FIG. 12b is the result of immunoblot analysis which shows that Arc protein synthesis rapidly induced by DHPG, which is an mGluR5 agonist, is inhibited by norathyriol.

FIG. 13 is diagrams of measuring the inhibitory efficacy of athyriol on mGluR-dependent long term depression formation in hippocampal brain slices. FIG. 13a is a diagram showing the change in fEPSP over time, and FIG. 13b is a diagram showing a graph which compares an average fEPSP value for the last 10 minutes with a 10-minute baseline and quantifies it.

FIG. 14 is diagrams of measuring the memory improvement and sociability enhancement effects of administration of athyriol in normal rats by a Y-type maze test and a 3-chamber test. FIG. 14a is the result of the Y-type maze test and FIG. 14b is the result of the 3-chamber test.

### MODE FOR INVENTION

Hereinafter, preferable examples are presented to help understanding of the present invention. However, the following examples are provided only to understand the present invention more easily, but the contents of the present invention are not limited by the examples.

### Example 1. Synthesis of 1,6,7-trihydroxy-3-methoxy-9H-xanthen-9-one (1,6,7-Trihydroxy-3-methoxy-9H-xanthen-9-on, athyriol, athyriol)

### Analysis Device

Devices used for confirmation of structures of the products obtained in the present invention are as below. For nuclear magnetic resonance spectrum (1H NMR), ADVANCE digital 500 was used, and as a solvent, CD₃OD or DMSO-d₆ was used. Mass spectrum was used, and represented in an m/z form.

### TLC and Column Chromatography

For TLC (Thin layer chromatography), silica gel (Merck F254) which is a product of Merk company was used, and for column chromatography, silica gel (Merck EM9385, 230-400 mesh) was used. In addition, in order to confirm substances isolated on TLC, a UV lamp (254 nm) was used, or they were confirmed by heating a plate, after soaking them in an anisaldehyde coloring reagent.

### Used Reagents

As reagents and solvents used in the present invention, Sigma-Aldrich and TCI products were purchased and used. Norathyriol used for athyriol synthesis was synthesized by the method of the prior patent KR10-2004245 (Preparing method of norathyriol using eco-friendly C-deglycosylation).

### Synthesis Method

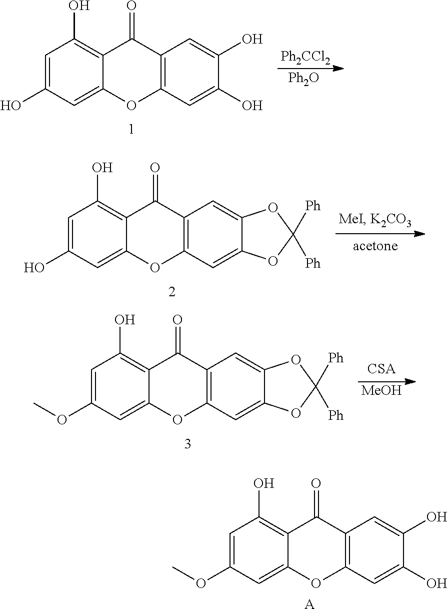

After adding Ph₂CCl₂ of 0.33 mL (1.73 mmol, 1.5 equivalents) to Ph₂O (10 mL) solution of norathyriol which is Compound 1 of 300 mg (1.15 mmol, 1 equivalent), they were heated and stirred at 175° C. for 2 hours. After cooling to a room temperature, solids obtained by recrystallizing them with hexane and filtering under reduced pressure were dried, and Compound 2 of 476 mg was obtained and immediately used for the next reaction.

To acetone solution of 2 of 130 mg (0.31 mmol, 1 equivalent) and K₂CO₃ of 53.9 mg (0.39 mmol, 1.3 equivalents), Mel of 0.03 mL (0.47 mmol, 1.5 equivalents) was added and stirred at a room temperature for 5 hours. The reaction solution was filtered to remove solids, and solvents were removed from filtrates under vacuum. The reaction mixture was purified by column chromatography using a 0.5% methanol solvent in dichloromethane to obtain Compound 3 of 87.5 mg (0.32 mmol, 100%).

Compound 3 of 87.5 mg (0.21 mmol, 1 equivalent) and CSA of 110.1 mg (0.47 mmol, 2.3 equivalents) were dissolved in MeOH and stirred at 55° C. for one day. The reactants were quenched with NaHCO₃ solution at a room temperature and extracted with ethyl acetate and water, and then washed with brine. Organic layers were dried with anhydrous MgSO₄, and solvents were removed under vacuum, and then the reaction mixture was purified by column chromatography (silica gel) using a 2.5% methanol solvent in dichloromethane to obtain athyriol, which is desired Compound A of 15 mg (0.044 mmol, 20%).

MS m/z 275 (M+H⁺)

¹H NMR (500 MHZ, DMSO) δ 13.18 (s, 1H), 7.38 (s, 1H), 6.87 (s, 1H), 6.57 (d, J=2.2 Hz, 1H), 6.33 (d, J=2.2 Hz, 1H), 5.76 (s, 1H), 3.86 (s, 3H).

### Experimental Example 1. Evaluation of In Vivo Efficacy of Athyriol on Autism Spectrum Disorder Model Pten KO Mice

In order to evaluate effects of athyriol on autism spectrum disorder symptoms, in particularly, specifically, effects on 1) memory and cognitive function disorder, 2) anxiety disorder, 3) sociability disorder and 4) macrocephaly, evaluation of in vivo efficacy of athyriol for an autism spectrum disorder, Pten KO mice, was performed.

### 1-1. Administration of Athyriol to Pten KO Mice

As Pten KO mice, Ptenf/f_cre/cre KO mice or Ptenf/f_cre/++ Het mice (KD) were obtained by mating Pten floxed/floxed mice and CAMKII-cre mice. DMSO or athyriol was intraperitoneally injected at 5 or 10 mg/kg for 2 weeks from the age of mice of 6 weeks, and a behavioral test was performed in 7 days after administration. Pten floxed/floxed mice were used as littermates of the same age as a normal control group.

### 1-2. Confirmation of Memory Ability Improvement Effect Through a Y-Type Maze Test

A Y-maze was performed in a Y-shaped structure maze made of black acrylic material in which an angle of each arm was 120°, and a light and a cam were installed on the ceiling, and it was checked that white mice entered each arm while the surroundings were covered with a curtain. Through this, short-term memory was analyzed. In order to adapt mice to the environment, they were acclimated to the experimental space for 30 minutes. The mice were placed at one end of the maze and allowed to move freely for 12 minutes. Accuracy was measured by dividing time into 8 minutes, 10 minutes and 12 minutes. When the four paws of the mice entered the entrance, it was considered as completely entered. The percentage of accuracy was calculated as the number of times which a mouse entered each maze without repetition. The number of times entering each of three different arms was divided by the total number of times entering arms-2 and converted into a percentage for datafication. All the results were statistically processed using ANOVA test.

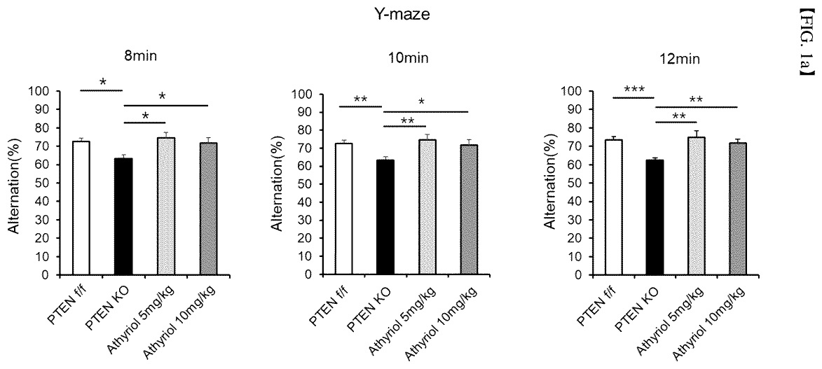

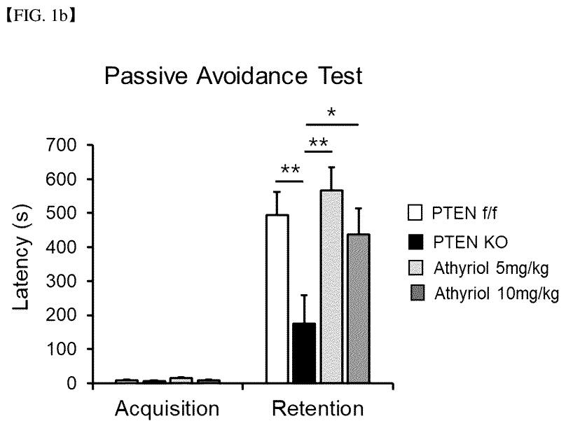

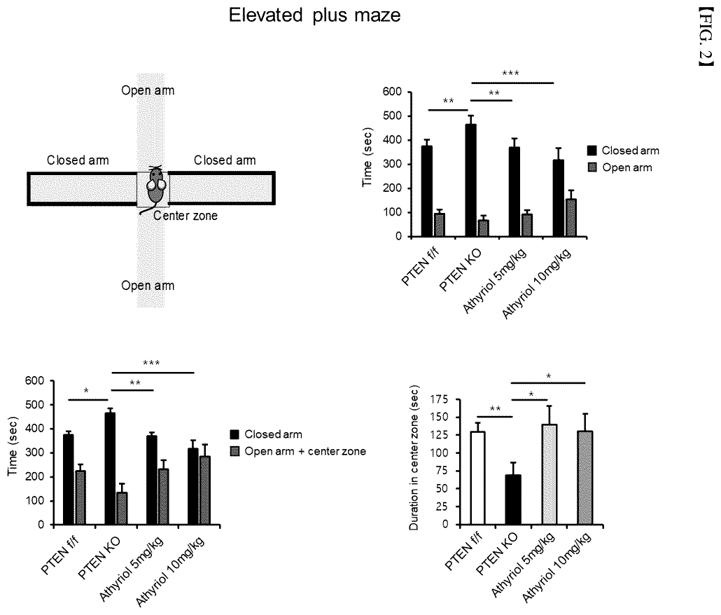

As a result, as shown in FIG. 1a, it was confirmed that in the Y-type maze test, the Pten KO mice showed short-term memory loss significantly compared to the control group, Pten floxed/floxed mice, but in the Pten KO mice in which athyriol was administered at a dose of 5 or 10 mg/kg for 14 days, the short-term memory was recovered almost to the level of the normal control group, Pten floxed/floxed mice.

### 1-3. Confirmation of Cognitive Function Improvement of Athyriol Through a Passive Avoidance Test

A passive avoidance test was performed as described in Heo et al. (J. Ethnopharmacol. (2009) 122:20-27). After training the avoidance action by strong light three times a day, an electric foot shock (1 mA, 300 g test) was applied for 3 seconds in a dark room at the same time on the next day. After placing white mice in the same room in 24 hours after applying the shock, an avoidance response by light, that is, the time to move from a bright room to a dark room was measured, and it was dataficated for each group. Exactly after 24 hours of adaptation training, the mice were put back into the bright room and the latency time required to enter the dark room was measured for 720 seconds.

As a result, as shown in FIG. 1b, also in the passive avoidance test, it was confirmed that the Pten KO mice had memory ability lowered to 36% or less compared to the control group mice, but in the Pten KO mice in which athyriol was administered at a dose of 5 mg/kg, the memory ability was rather more excellent than the control group or in the Pten KO mice in which it was administered at a dose of 10 mg/kg, it was recovered at a level of 89% of the control group.

Therefore, through Experimental examples 1-2 and 1-3, it could be confirmed that the memory and cognitive functions in the Pten KO mice which were lowered compared to the normal group were recovered at a level of the normal group by administration of athyriol.

### 1-4. Elevated Plus Maze Test Anxiety Disorder Animal Model Behavioral Test

In order to confirm whether emotional instability was improved, when athyriol was administered to an autism spectrum disorder animal model, Pten KO mice, an elevated plus maze test was performed. An anxiety disorder animal model behavioral test was evaluated as a tendency to avoid an open arm exposed to the elevated-plus maze and stay in a closed arm. Analysis was conducted with a video tracking system (Ethovision EPM program, Noldus information technology, Wageningen, Netherlands). In the elevated plus maze test measuring emotional instability disorder, when the emotional anxiety increases, the time spent in the closed arm increases compared to the time spent in the open arm.

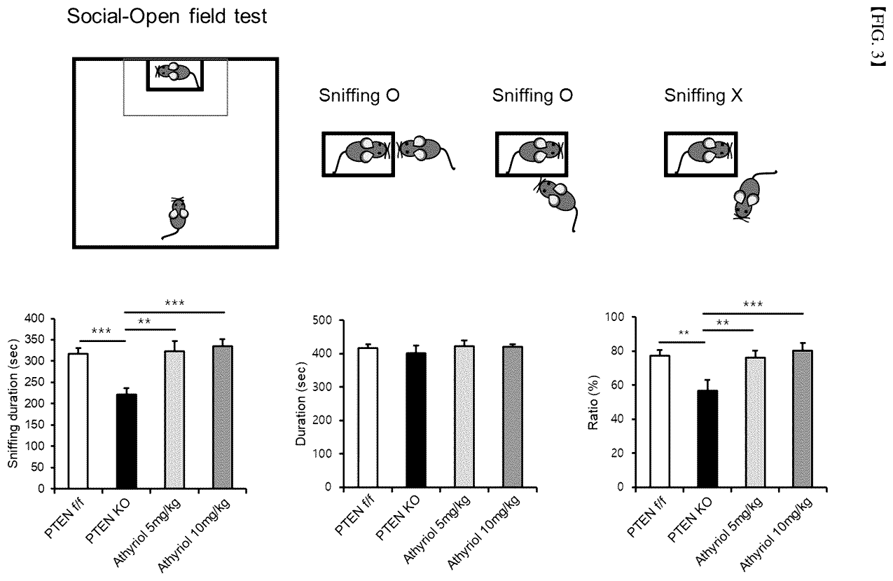

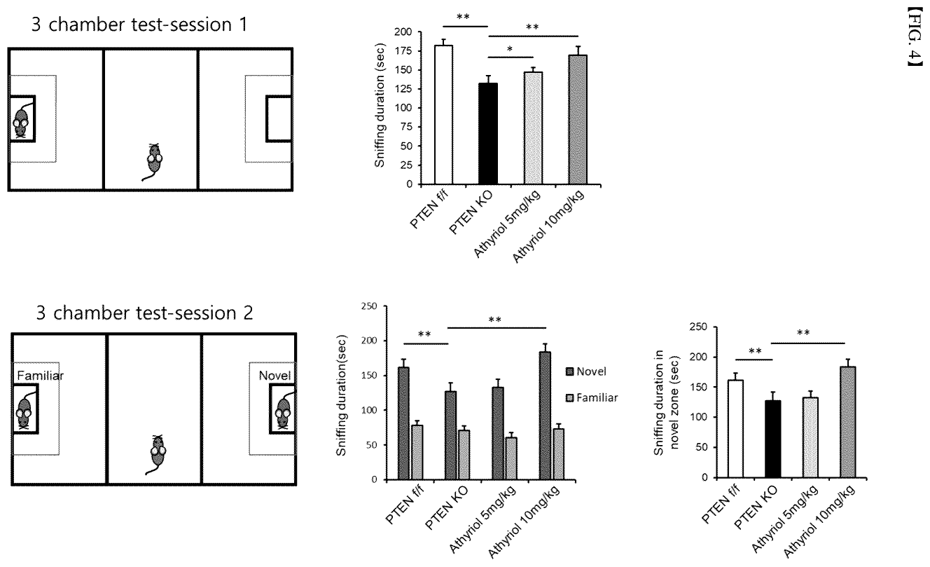

As a result, as shown in FIG. 2, in the Pten KO mice, the time spent in the closed arm increases compared to the time spent in the open arm by 24% than the normal control group, and therefore, it shows that emotional anxiety increases by Pten KO. However, in the Pten KO mice in which athyriol was administered, the time spent in the closed arm was reduced by 15% to the normal control group level (2% decrease) or less. In addition, the time in the center zone which is the center of the open arm was also reduced about twice compared to the normal control group in the Pten KO mice, but it was recovered to the normal control group level in the Pten KO mice in which athyriol was administered. Accordingly, it could be confirmed that the anxiety disorder occurring in the Pten KO mice was improved to the normal group level by athyriol administration.

### 1-5. Social-Open Field Test

When athyriol was administered to the Pten KO mice, in order to confirm the sociability disorder was improved, a social-open field test was performed. After adapting the Pten-KO mice to the record environment for 30 minutes, they were adapted to the open filed test space made of black acrylic for 10 minutes. An unfamiliar mouse was placed in a test space in a transparent acrylic cage allowing minimal contact with the sense of smell. The movement of the test mouse was recorded for 10 minutes and analyzed with ethovision 3.1 program. Then, sociability was measured by setting 10 cm around the cylinder where the unfamiliar mouse was placed.

The social-open field test is measuring sociability as the time spent in a social interaction zone in contact with other mice in an open space, and measures the time of actually contacting also in the contact space, or sniffling toward other mice. In this measurement, in the social interaction zone, the time facing other directions was excluded from the direct counterpart searching time.

As a result, as shown in FIG. 3, the direct counterpart searching time of the Pten KO mice was reduced to about 77% of the searching time of the normal control group. However, in the Pten KO mice to which athyriol was administered at a dose of 5 or 10 mg/kg, the direct counterpart searching time increased all by 6% to the normal control group level (2% increase) or more. Therefore, it could be confirmed that sociability reduced by Pten KO was recovered by athyriol administration.

### 1-6. 3-Chamber Test

3-chamber test is a test to confirm sociability, social cognition, and social sexual preference, and it was tested by composing sessions 1 and 2. The experimental site was consisted of three chambers made of transparent acrylic walls with a small door and each chamber was length 50 cm×width 100 cm×height 50 cm. Through a cylindrical cage, the sense of smell and minimal contact of the test mice to the unfamiliar mouse were allowed. Prior to the test, the test mice were placed in an intermediate chamber and adapted for 5 minutes with both doors closed. The test was performed by dividing into sessions I and II. In the 3-chamber sociability measurement-1 session, an unfamiliar mouse met first was placed in a transparent box located at one end of the three rooms, and an empty transparent box of the same size was placed in the room located at the other side, and the target mouse was placed in the middle room, and then the time searching toward the counterpart within the social interaction zone was measured.

As a result, as shown in FIG. 4, in the Pten KO mice, the time searching the unfamiliar mouse was significantly reduced compared to the normal control group, and it was recovered to the normal control group level in a concentration-dependent manner by athyriol administration. In the 3-chamber sociability measurement-2 session, an unfamiliar met first was placed in a transparent box located at one end of the three rooms, and the mouse met in the session 1 was placed in the same transparent box in the room located at the other side. As a result of measuring the time to search the counterpart within the social interaction zone, in the Pten KO mice, the total interaction time of searching the unfamiliar mouse and familiar mouse was reduced compared to the control group, and in particular, the time of searching the unfamiliar mouse was significantly reduced by 21%. However, in the Pten KO mice in which athyriol was administered at a dose of 5 or 10 mg/kg, the time of searching the unfamiliar mouse increased in a dose-dependent manner, and in the Pten KO mice in which it was administered at a dose of 10 mg/kg, it was increased by 14% than that of the normal control group. Therefore, it could be confirmed that the sociability disorder occurring in the Pten KO mice was improved to the normal group level by athyriol administration.

1-7. Confirmation of Macrocephaly Alleviation Effect of Pten KO Mice when Athyriol is Treated

When athyriol was administered into Pten KO mice, whether the weight of brain increased compared to a mouse without Pten knockout was reduced was confirmed. In other words, whether athyriol had efficacy of alleviating macrocephaly symptoms was confirmed through the following experiment.

Specifically, the body weight was measured after the aforementioned behavioral test of Experimental examples 1-2 to 1-6, and then animals were sacrificed. After removing the skull, the brain including forebrain, midbrain and hindbrain was extracted and the ratio of brain weight to body weight and the body weight were measured.

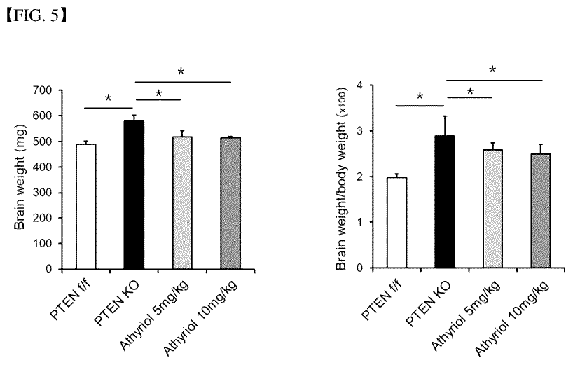

As a result, as shown in FIG. 5, the average brain weight of 8-week-old Pten KO mice was 580.03 g, which increased by 18.8% compared to the average brain weight of 488.43 g of the control group without Pten knockout, and the brain weight of the Pten KO mice in which athyriol was administered at a dose of 5 or 10 mg/kg for 14 days was measured in average as 517 g and 515 g, respectively. Therefore, it was confirmed that athyriol alleviated macrophaly symptoms of the Pten KO mice by 68% or more. Even in the case of the numerical value of the brain weight corrected by the body weight, in the Pten KO mice, it increased by 1.4 times or more compared to the control group, but in the Pten KO mice in which athyriol was administered at 5 or 10 mg/kg, it increased just by about 1.3 times and 1.26 times, respectively, and therefore, it could be confirmed that the macrophaly symptoms of the Pten KO mice were alleviated by athyriol administration.

### Experimental Example 2. In Vitro Kinase Assay Evaluation for mTORC2 Activity Inhibitory Efficacy of Norathyriol and Athyriol

2-1. In Vitro mTORC2 Kinase Assay for mTORC2 Activity Inhibitory Efficacy of Norathyriol and Athyriol

In order to investigate whether norathyriol and athyriol directly inhibit mTORC2, in vitro mTORC2 kinase assay was performed. After introducing Flag-mLST8 into a human cell line 293T, mTORC was isolated by co-immunoprecipitation using Flag antibody. In the in vitro mTORC2 kinase assay, the activity of the isolated mTORC2 was evaluated by measuring phosphorylation of GST-Akt Ser473, which is a selective substrate of mTORC2 by immunoblot analysis. In the control group in which Flag-mLST8 was not introduced, GST-Akt Ser473 phosphorylation was almost not measured. On the other hand, in the experimental group in which Flag-mLST8 was introduced, GST-Akt Ser473 phosphorylation increased significantly. In addition, this increased GST-Akt Ser473 phosphorylation was mostly reduced by AZD8055, which is a dual mTOR inhibitor. Therefore, it was judged that GST-Akt Ser473 phosphorylation measured in the in vitro mTORC2 kinase assay was generated by mTORC2. In order to evaluate the inhibitory effect of norathyriol and athyriol on mTORC2 activity, norathyriol or athyriol was pretreated for 15 minutes, and then in vitro mTORC2 kinase assay was carried out.

More specifically, the HEK 293T cells were cultured in a DMEM culture medium in which 10% heat inactivated FBS+1× Glutamax was added. 8×10⁵ HEK 293T cells were seeded in a 100 mm culture dish, and then they were cultured for 1 day. After mixing 42 μl Lipofectamine, 21 μl plus reagent, and 21 μg Flag-mLST8 plasmid DNA to 1 ml OPTI-MEM, it was shaken at a room temperature for 25 minutes to prepare a transfection DNA mixture. The transfection DNA mixture was slowly treated on the cells drop by drop, and after culturing for 5 hours, the culture medium was replaced with a new culture medium. After additional culturing for 48 hours, the cells were hemolyzed with CHAPS buffer (50 mM HEPES (pH7.4), 100 mM NaCl, 2 mM EDTA, 0.3% CHAPS, 10 mM sodium pyrophosphate, 10 mM sodium β-glycerophosphate, 1 mM PMSF, 1 μg/ml leupeptin, 1 μg/ml pepstatin A, 10 nM aprotinin, 1 mM Na₃VO₄, 10 nM calyculin A), and centrifuged at 4° C. at 13,000 rpm for 10 minutes to obtain the supernatant and acquire cell lysate. The protein concentration of the cell lysate was determined with BCA protein assay kit (Thermo Fisher Scientific). After adding anti-Flag M2 affinity gel (50% slurry, Sigma) of 30 μl to the cell lysate of 1 mg, it was shaken at 4° C. for 3 hours. After that, affinity gel was washed with CHAPS buffer 3 times and kinase buffer (50 mM HEPES (pH7.5), 2 mM DTT, 10 mM MnCl₂, 10 mM MgCl₂) for washing once. After adding kinase buffer [-substrate-ATP] (50 mM HEPES (pH7.5), 2 mM DTT, 10 mM MnCl₂, 10 mM MgCl₂, 10 nM calyculin A) of 10 μl to the affinity gel, it was shaken at 30° C. for 15 minutes. After that, kinase buffer (50 mM HEPES (pH7.5), 2 mM DTT, 10 mM MgCl₂, 10 mM MnCl₂, 1 mM ATP, 500 ng GST-AKT, 10 nM calyculin A) of 10 μl was added and shaken at 30° C. for 45 minutes to progress a kinase reaction. 4× SDS sample buffer of 14 μl was added to the affinity gel, and then it was boiled at 95° C. for 5 minutes to stop the reaction. Ser473 phosphorylation of GST-AKT, which is a substrate of mTORC2, was measured by performing immunoblot analysis using AKT antibody (#4691, Cell signaling) and phospho Ser473 AKT antibody (#4060, Cell signalig). In order to measure the mTORC2 activity inhibitory effect by the compound, both the kinase buffer [-substrate-ATP] and the kinase buffer included the compound.

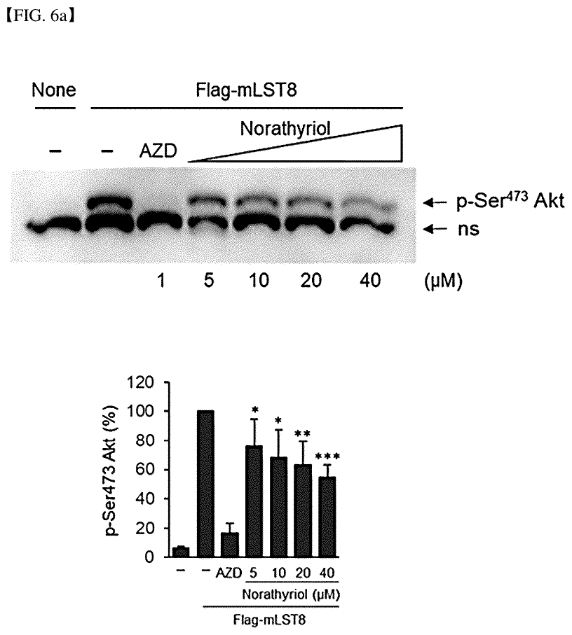

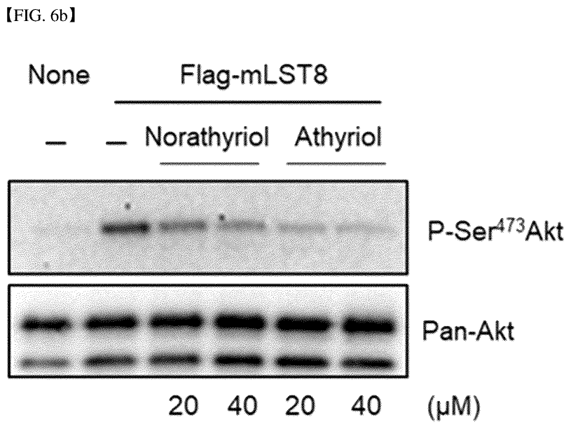

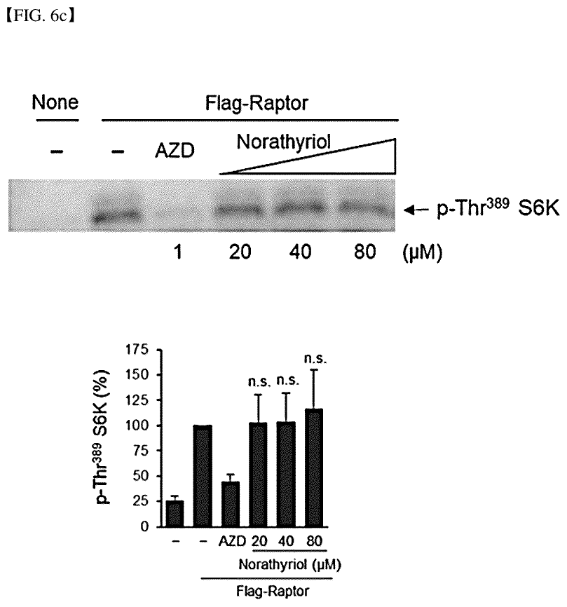

As a result, as shown in FIG. 6a, it could be confirmed that GST-Akt Ser473 phosphorylation by mTORC2 was reduced in a concentration-dependent manner by norathyriol. Norathyriol reduced GST-Akt Ser473 phosphorylation in 5 μM by 20% or more in average, and in 40 μM, it reduced by 50%. Therefore, IC_{50,mTORC2} of norathyriol was evaluated as 40 μM.

In addition, as shown in FIG. 6b, as the result of comparing and evaluating the mTORC2 inhibitory efficacy of norathyriol and athyriol in the in vitro mTORC2 kinase assay, norathyriol inhibited the mTORC2 activity by 46% in 40 μM, and athyriol inhibited the mTORC2 activity by 59% in 20 μM and 67% in 40 μM.

2-1. In Vitro mTORC1 Kinase Assay for mTORC1 Activity Inhibitory Efficacy of Norathyriol

In order to investigate the effect on mTORC1 activity of norathyriol, in vitro mTORC1 kinase assay was performed. After introducing Flag-Raptor into a human cell line 293T, mTORC1 was isolated by co-immunoprecipitation using Flag antibody. In addition, the activity of the isolated mTORC1 complex was evaluated by measuring phosphorylation of GST-S6K Thr389, which is a selective substrate of mTORC1, by immunoblot analysis. In the control group in which Flag-Raptor was not introduced, GST-S6K Thr389 phosphorylation was almost not measured. On the other hand, in the experimental group in which Flag-Raptor was introduced, GST-S6K Thr389 phosphorylation increased significantly. In addition, this increased GST-S6K Thr389 phosphorylation was mostly reduced by AZD8055, which is a dual mTOR inhibitor. Therefore, it was judged that GST-S6K Thr389 phosphorylation measured in the in vitro mTORC1 kinase assay was generated by mTORC1. In order to evaluate the inhibitory effect of norathyriol on mTORC1 activity, norathyriol was pretreated for 15 minutes, and then in vitro mTORC1 kinase assay was carried out.

More specifically, the HEK 293T cells were cultured in a DMEM culture medium in which 10% heat inactivated FBS+1× Glutamax was added. 8×10⁵ HEK 293T cells were seeded in a 100 mm culture dish, and then they were cultured for 1 day. After mixing 42 μl Lipofectamine, 21 μl plus reagent, and 21 μg Flag-Raptor plasmid DNA to 1 ml OPTI-MEM, it was shaken at a room temperature for 25 minutes to prepare a transfection DNA mixture. The transfection DNA mixture was slowly treated on the cells drop by drop, and after culturing for 5 hours, the culture medium was replaced with a new culture medium. After additional culturing for 48 hours, the cells were hemolyzed with CHAPS buffer (50 mM HEPES (pH7.4), 100 mM NaCl, 2 mM EDTA, 0.3% CHAPS, 10 mM sodium pyrophosphate, 10 mM sodium β-glycerophosphate, 1 mM PMSF, 1 μg/ml leupeptin, 1 μg/ml pepstatin A, 10 nM aprotinin, 1 mM Na₃VO₄, 10 nM calyculin A), and centrifuged at 4° C. at 13,000 rpm for 10 minutes to obtain the supernatant and acquire cell lysate. The protein concentration of the cell lysate was determined with BCA protein assay kit (Thermo Fisher Scientific). After adding anti-Flag M2 affinity gel (50% slurry, Sigma) of 30 μl to the cell lysate of 1 mg, it was shaken at 4° C. for 3 hours. After that, affinity gel was washed with CHAPS high salt buffer (50 mM HEPES (pH7.4), 500 mM NaCl, 2 mM EDTA, 0.3% CHAPS) 3 times and kinase buffer for washing (25 mM HEPES-KOH (pH7.4), 20 mM KCl) once. After adding kinase buffer [-substrate-ATP] (25 mM HEPES-KOH (pH7.4), 50 mM KCl, 10 mM MgCl₂, 10 nM Calyculin A) of 10 μl to the affinity gel, it was shaken at 30° C. for 15 minutes. After that, kinase buffer (25 mM HEPES-KOH (pH7.4), 50 mM KCl, 10 mM MgCl₂, 500 μM ATP, 100 ng GST-S6K1, 10 nM Calyculin A) of 10 μl was added and shaken at 30° C. for 45 minutes to progress a kinase reaction. 4× SDS sample buffer of 14 μl was added to the affinity gel, and then it was boiled at 95° C. for 5 minutes to stop the reaction. Thr389 phosphorylation of GST-S6K1, which is a substrate of mTORC1, was measured by performing immunoblot analysis using phospho Thr389 p70 S6 kinase antibody (#9205S, Cell Signaling) and GST antibody (#A190-122A, Bethyl Laboratories). In order to measure the mTORC1 activity inhibitory effect by the compound, Both the kinase buffer [-substrate-ATP] and the kinase buffer included the compound.

As a result, as shown in FIG. 6c, differently from the tendency of the change in GST-Akt Ser473 phosphorylation in the in vitro mTORC2 kinase assay, phosphorylation of GST-S6K Thr389, which is a substrate of mTORC1, was not reduced at all by norathyriol. Even not only in the experimental groups in which norathyriol was treated by 20 μM and 40 μM, but also in the experimental group in which it was treated by 80 μM, GST-S6K Thr389 phosphorylation was not reduced at a significant level. Therefore, IC_{50,mTORC1} of norathyriol was evaluated as >80 μM.

Thus, from the results of Experimental example 2-1 and Experimental example 2-2, it could be confirmed that norathyriol and athyriol are selective mTORC2 inhibitors which inhibits mTORC2 activity, but does not inhibit mTORC1, and it could be confirmed that athyriol had more excellent mTORC2 inhibitory efficacy compared to norathyriol.

### Experimental Example 3. Evaluation of mTORC2 Signaling Inhibitory Efficacy in Cells by Norathyriol

3-1. Confirmation of Norathyriol Effect on mTORC2 Signaling Pathway by Immunoblot Analysis

In order to investigate the effect on the mTORC2 signaling pathway of norathyriol in a culture cell model, after treating norathyriol to a human cell line A549, Akt Ser473 phosphorylation, Akt Thr308 phosphorylation, and NDRG1 Thr346 phosphorylation were measured by immunoblot analysis over time. The A549 cells were cultured in RPMI culture medium in which 10% heat inactivated FBS and 1× GlutaMax were added. After seeding the A549 cells of 2×10⁵ in a 60 mm culture dish and culturing them for 1 day, norathyriol was treated to the cells. The cells were washed with ice-cold 1× PBS twice, and then the cells were hemolyzed using RIPA buffer (50 mM Tris-HCl (pH7.4), 150 mM NaCl, 0.25% sodium deoxycholate, 1% NP-40, 1% SDS, 1 mM EDTA, 1 mM EGTA, 1 mM sodium β-glycerophosphate, 1 mM PMSF, 1 μg/ml leupeptin, 1 μg/ml pepstatin A, 10 nM aprotinin, 1 mM Na₃VO₄, 50 mM NaF, 10 nM calyculin A) on ice. After removing undissolved substances by centrifuging at 4° C. at 15,000× g for 15 minutes, the supernatant was recovered to determine the protein concentration with BCA protein assay kit (Thermo Fisher Scientific). 2× SDS-PAGE sample buffer in the same volume was added to the supernatant and boiled for 5 minutes, and then it was developed under a reducing condition by SDS-polyacrylamide gel electrophoresis. The developed protein was transferred into a PVDF membrane by an electro blotting method. The membrane was blocked in TBST containing 5% skim milk (100 mM Tris-Cl, pH 8.8, 150 mM NaCl, 0.1% Tween-20) at a room temperature for 1 hour, and then it was reacted with a primary antibody. After washing with TBST for 10 minutes 3 times, it was reacted with a secondary antibody conjugated with HRP. After washing with TBST for 10 minutes 3 times, it was reacted with an enhanced chemiluminescence (ECL) reagent to obtain an image of the protein combined with the secondary antibody. For the immunoblot analysis, AKT antibody (#4691, Cell Signaling), phospho-Ser473 AKT antibody (#4060, Cell Signaling), phospho-Thr308 AKT antibody (#2965, Cell Signaling), p70 S6 kinase antibody (#9202, Cell Signaling), and phospho-Thr389 p70 S6 kinase antibody (#9205, Cell Signaling) were used.

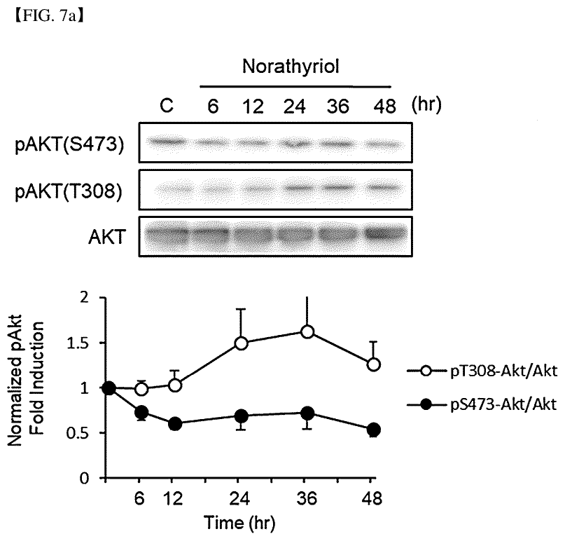

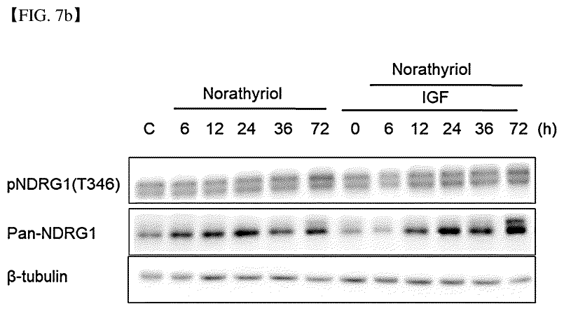

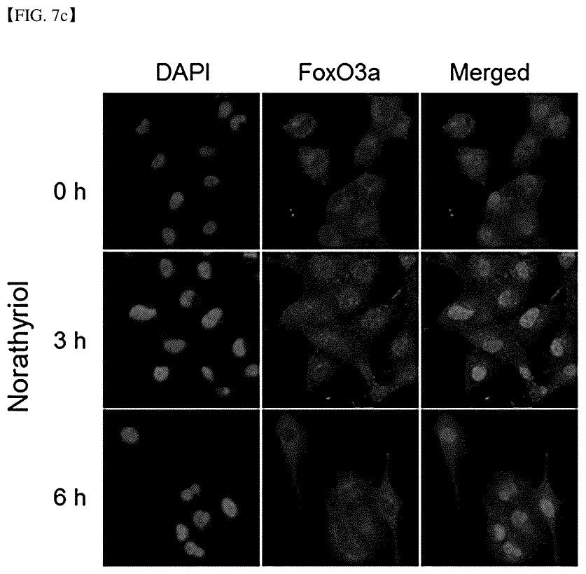

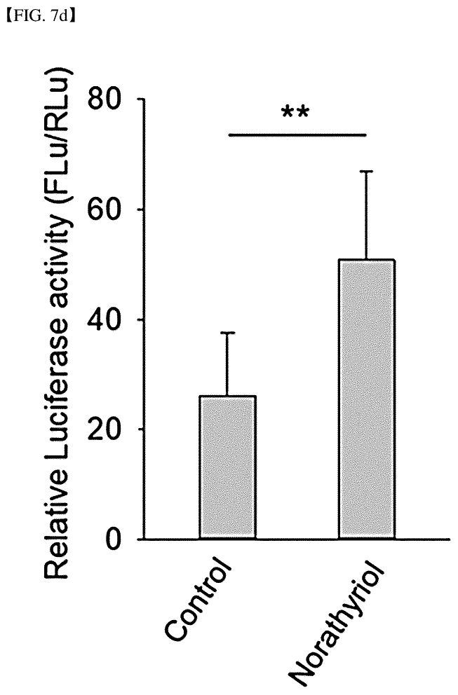

As a result, as shown in FIG. 7a, it was confirmed that by norathyriol, Akt Ser473 phosphorylation which is an mTORC2 activity indicator was reduced, whereas Akt Thr308 phosphorylation which is a PDK1 activity indicator was not reduced and rather increased after 24 hours. As Akt has complete activity when Ser473 is phosphorylated, the Akt activity is reduced when Akt Ser473 phosphorylation is reduced, and the mTORC1 activity which is its downstream transducer is subsequently reduced. It is judged that at the beginning of treatment with norathyriol, mTORC2 is inhibited and Akt Ser473 phosphorylation is reduced, but after that, PDK1 is activated by negative feedback by mTORC1 inhibition, and therefore, Akt Thr308 phosphorylation increases from 24 hours.

mTORC2 activates SGK1, and SGK1 phosphorylates NDRG1 Thr346. NDRG1 phosphorylated at Thr346 is removed by a ubiquitination-proteasome system. Accordingly, when the mTORC2 activity is inhibited, NDRG1 Thr346 phosphorylation is reduced, whereas NDRG1 protein increases. As shown in FIG. 7b, it could be confirmed that after treating norathyriol to the A549 cells, the NDRG1 protein increased significantly, and at the same time, the ratio of NDRG1 Thr346 phosphorylation to NDRG1 protein was reduced.

3-2. Confirmation of mTORC2 Signaling Inhibitory Effect of Norathyriol by Immunofluorescent Staining Analysis

mTORC2 activates SGK3, and SGK3 phosphorylates Fox03a, allowing Fox03a to remain in the cytoplasm. When mTORC2 is inhibited, Fox03a is dephosphorylated and translocated to the nucleus, and in the nucleus, it binds to FHRE located in a gene promoter to induce gene expression. Therefore, the change in the intracellular location of Fox03a and FHRE dependent gene expression become indicators for evaluating mTORC2 activity. In order to evaluate the effect of norathyriol on the mTORC2 activity, norathyriol was treated to A549, and then the change in the intracellular location of Fox03a was investigated by immunofluorescent staining.

Specifically, the A549 cells of 1×10⁵ were seeded in a confocal dish coated with 35 mm collagen and cultured for 1 day. After treating norathyriol, the cells were fixed with 4% paraformaldehyde at 4° C. for 15 minutes. After treating PBS containing 0.5% Triton X-100 (PBS-T) at a room temperature for 5 minutes for permeabilization, PBS containing 5% normal goat serum and 1% gelatin was treated at a room temperature for 30 minutes for blocking. The Fox03a antibody diluted in 1% BSA was treated at a room temperature for 1 hour. After washing the culture dish with PBS-T, an FITC conjugated anti-rabbit secondary antibody diluted in 1% BSA was treated at a room temperature for 50 minutes. The nucleus was stained by treating DAPI by 100 ng/ml at a room temperature for 3 minutes. After washing with PBS-T, mounting was performed using mounting solution containing p-phenylenediamine, and then it was observed with a confocal microscope.

As a result, as shown in FIG. 7c, it could be confirmed that Fox03a located in the cytoplasm before treating norathyriol moved to the nucleus when norathyriol was treated for 3 hours.

3-3. Confirmation of mTORC2 Signaling Inhibitory Effect of Norathyriol by Luciferase Reporter Analysis

mTORC2 activates SGK3, and SGK3 phosphorylates Fox03a, allowing Fox03a to remain in the cytoplasm. When mTORC2 is inhibited, Fox03a is dephosphorylated and translocated to the nucleus, and in the nucleus, it binds to FHRE located in a gene promoter to induce gene expression. Therefore, the change in the intracellular location of Fox03a and FHRE dependent gene expression become indicators for evaluating mTORC2 activity. In order to evaluate the effect of norathyriol on the mTORC2 activity, norathyriol was treated to A549, and then expression of an FHRE-synthetic luciferase gene was investigated by luciferase reporter analysis.

Specifically, A549 cells of 2×10⁵ were seeded in a 35 mm culture dish and cultured for 1 day, and then a 2.5 μg FHRE-Luc plasmid and a 0.1 μg renilla luciferase plasmid were introduced by a transfection method using Lipofectamine LTX reagent (Thermofisher). After 18 hours, norathyriol was treated for 24 hours. Luciferase assay was performed with Dual Luciferase Reporter Assay System (Promega) according to the manufacturer's instructions.

As a result, as shown in FIG. 7d, it could be confirmed that expression of the FHRE-synthetic luciferase gene increased by twice or more by norathyriol.

From the experimental result of Experimental example 3-1 to Experimental example 3-3, it could be confirmed that norathyriol inhibited the mTORC2 signaling pathway in the cells.

### Experimental Example 4. Evaluation of De Novo mTORC2 Complex Formation Inhibitory Efficacy by Norathyriol

In the in vitro kinase assay of Experimental example 2, it could be confirmed that norathyriol directly inhibited the mTORC2 activity. In case of a competitive inhibitor for an mTOR catalytic activity site or an allosteric inhibitor, the effect on mTORC2 complex formation may be slight, but in case of a protein-protein interaction modulator, the possibility to inhibit mTORC2 complex formation is large. In order to evaluate the effect on de novo mTORC2 complex formation of norathyriol, Flag-Protor1 was introduced to A549, and then the amount of mTORC2 formed together with Flag-Protor1 by co-immunoprecipitation using Flag antibody was investigated by immunoblot analysis.

Specifically, the A549 cells were cultured in RPMI culture medium in which 10% heat inactivated FBS and 1× GlutaMax were added. The A549 cells of 1.6×10⁶ were seeded in a 100 mm culture dish, and then cultured for 1 day. After mixing 56 μl Lipofectamine (#15338-100, Invitrogen), 20.4 μl plus reagent, and 20.4 μg plasmid DNA to 4 ml OPTI-MEM, it was shaken at a room temperature for 25 minutes to prepare a transfection DNA mixture. The transfection DNA mixture was slowly treated to the cells drop by drop, and the culture medium after culturing for 5 hours was replaced with a new culture medium. After culturing for 1 day, norathyriol was treated at a concentration of 30 μM for 24 hours. The cells were washed with ice-cold 1× PBS twice, and then cell lysate was obtained using CHAPS buffer of 1 ml (50 mM HEPES (pH7.4), 100 mM NaCl, 2 mM EDTA, 0.3% CHAPS, 10 mM sodium pyrophosphate, 10 mM sodium β-glycerophosphate, 1 mM PMSF, 1 μg/ml leupeptin, 1 μg/ml pepstatin A, 10 nM aprotinin, 1 mM Na₃VO₄) on ice. After adding 40 μl anti-Flag M2 affinity gel (#A2220, Sigma, 50% slurry) to the 1 mg cell lysate, it was shaken at 4° C. for 3 hours. After that, affinity gel was washed with 500 μl CHAPS buffer 3 times, and then 50 μl 2× SDS-PAGE sample buffer was added to the affinity gel to recover immunoprecipitates. For immunoblot analysis, mTOR antibody (#2972, Cell Signaling), Rictor antibody (#2114, Cell Signaling), Raptor antibody (#SC-81537, Santa Cruz), mSin1 antibody (#A300-910A, Bethyl), Flag-M2 antibody (#F1804, Sigma), Protor1 antibody (#ab185995, Abcam), and GβL (mLST8) antibody (#3227, Cell Signaling) were used.

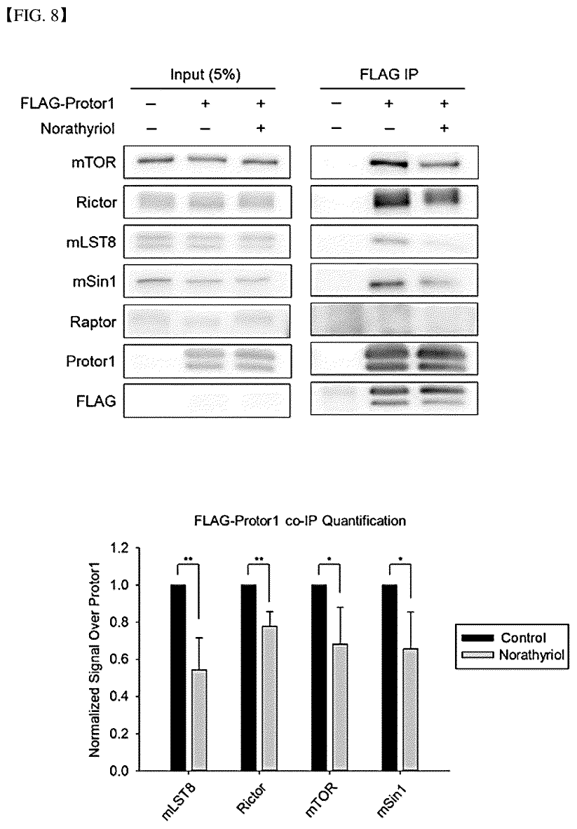

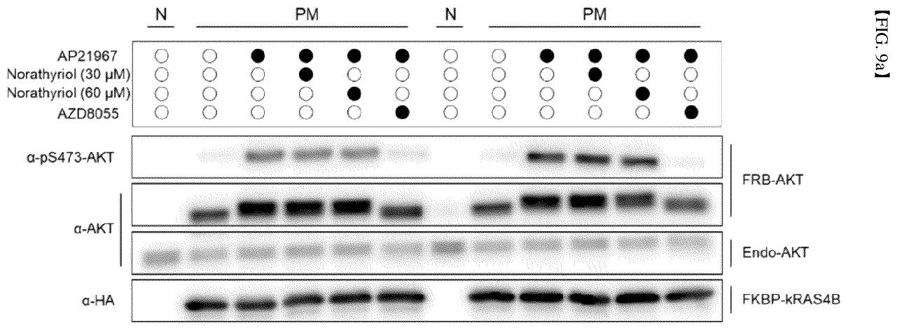

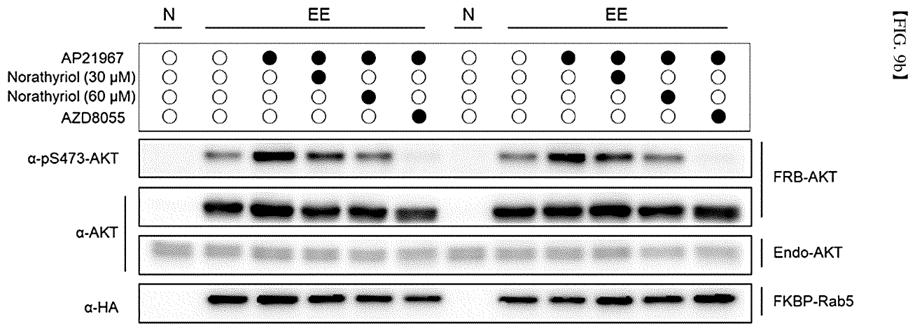

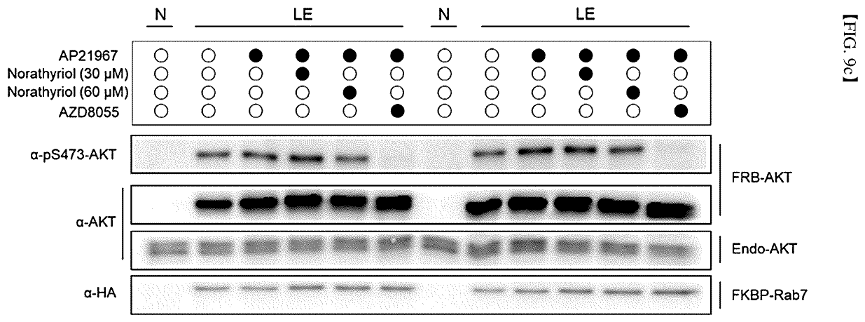

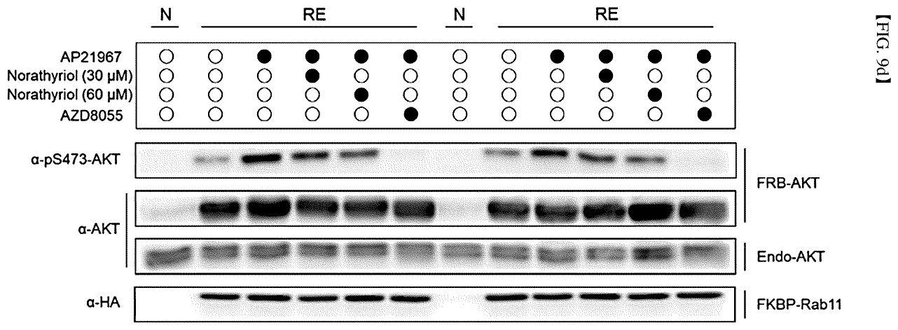

As a result, as shown in FIG. 8, it could be confirmed that mTOR, Rictor, mLST8, and mSin1, which are mTORC2 structural proteins forming a complex with Flag-Protor1, were reduced at a significant level by norathyriol. The amount of the mTORC2 structural proteins in the cell lysate was similar in the control group in which a drug was not treated and the experimental group in which norathyriol was treated, and the Protor1 amount precipitated by Flag antibody was also similar. However, mTOR, Rictor, mLST8, and mSin1, which are mTORC2 structural proteins precipitated with Protor1, were reduced by 25˜45% in the experimental group in which norathyriol was treated compared to the control group. From this experimental result, it could be confirmed that norathyriol inhibited de novo mTORC2 complex formation.

### Experimental Example 5. Analysis of Endosome Location Selective mTORC2 Inhibitory Effect of Norathyriol

mTORC2 is located in the cell membrane, endosome, ER and mitochondrial membrane. mTORC2 forms an mTORC2 complex as an activated form of ras (ras-GTP) operates as a scaffold in the cell membrane, whereas it forms an mTORC2 complex as mLST8 operates as a scaffold in the endosome. By this difference, the mTORC2 inhibitory effect of norathyriol may be shown differently depending on the subcellular compartment where mTORC2 is located. In order to investigate the mTORC2 inhibitory effect of norathyriol depending on the subcellular compartment location, in vivo LocaTOR2 assay was performed (Ebner et al. (2017) J. Cell Biol. 216:343-353). After introducing FKBP-recruiter selectively located in the membrane of the subcellular compartment and FRB^{T2098L}-AKT2 which is an mTORC2 substrate, AP21967 was treated for 40 minutes to induce binding between the FKBP-recruiter and FRB-AKT2, and FRB-AKT2 was moved to a specific subcellular compartment. In addition, the degree of phosphorylation of Ser473 of FRB-Akt by mTORC2 located in the site thereof was investigated by immunoblot analysis.

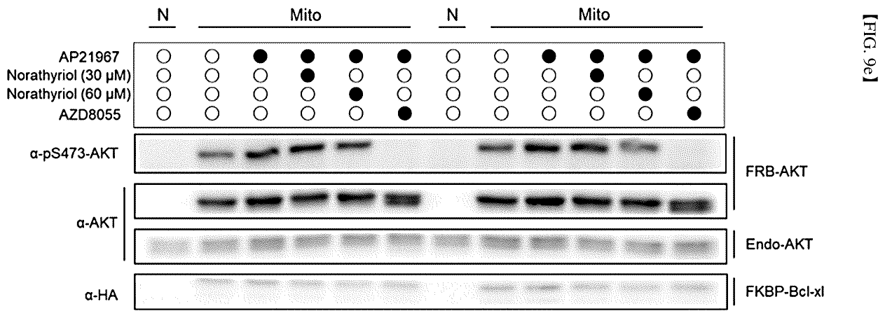

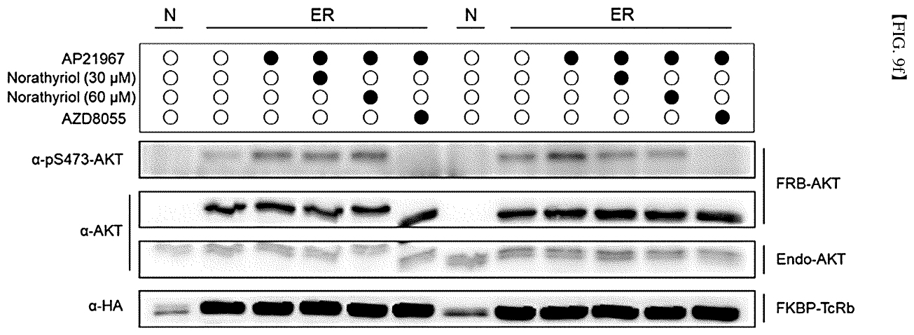

As a result, as shown in FIG. 9a, when FRB-AKT2 was moved to the cell membrane by treating AP21967 to the A549 cells in which KRas4B^{C30}-FKBP and FRB-AKT2 were introduced, phosphorylation of FRB-AKT2 Ser473 increased, and FRB-AKT2 Ser473 phosphorylation increased like this was not reduced by norathyriol at concentrations of 30 μM and 60 μM. In contrast thereto, ADZ8055, which is a dual mTOR inhibitor, reduced the phosphorylation of FRB-AKT2 Ser473 to a level of the control group in which AP21967 was not treated. From this experimental result, it could be confirmed that norathyriol did not inhibit mTORC2 located in the cell membrane.

In addition, as shown in FIG. 9b, when FRB-AKT2 was moved to the early endosome by treating AP21967 to the A549 cells in which Rab5-FKBP and FRB-AKT2 were introduced, FRB-AKT2 Ser473 phosphorylation was reduced at a significant level by norathyriol.

Furthermore, as shown in FIG. 9c, when FRB-AKT2 was moved to the late endosome by introducing Rab7-FKBP as FKBP-recruiter, FRB-AKT2 Ser473 phosphorylation was reduced at a significant level by norathyriol.

Moreover, as shown in FIG. 9d, when FRB-Akt2 was moved to the recycling endosome by introducing Rab11-FKBP as FKBP-recruiter, FRB-AKT2 Ser473 phosphorylation was reduced at a significant level by norathyriol.

In addition, as shown in FIGS. 9e and 9f, phosphorylation of FRB-AKT2 Ser473 induced by AP21967 was reduced by norathyriol in the cells in which Bcl-XL-FKBP and TcRb-FKBP were introduced as FKBP-recruiter.

As shown in FIG. 9g, which is a drawing of quantifying a to f of FIG. 9, IC_{50, mTORC2, PM} was measured as >60 μM, and IC_{50, mTORC2, EE} was <30 μM, and IC_{50, mTORC2, LE} was <30 μM, and IC_{50, mTORC2, RE} was 40 μM. Accordingly, it could be confirmed that norathyriol had endosome location selectivity which inhibits mTORC2 located in the early endosome, late endosome, and recycling endosome, but does not inhibit mTORC2 located in the cell membrane.

### Experimental Example 6. Evaluation of Excessive Dendritic Arborization Inhibitory Efficacy by Norathyriol in CYFIP1 Overexpression Autism Spectrum Disorder Culture Cell Model

Neural stem cells were primarily cultured from the hippocampal vestigial region of white mouse E16 embryos, and CYFIP1, which was found as a gene causing autism spectrum disorder, was introduced by transfection and overexpressed. When the neural stem cells in which CYFIP1 was overexpressed were differentiated, the neurite length and branch number increased compared to the normal cell, the number of dendritic spine increased, and morphological changes such as an increase in rod-shaped dendritic spines with a size of 0.6 μm or more, which are larger than the normal size, were accompanied. Therefore, CYFIP1 overexpression was used as a culture cell model of autism.

By treating norathyriol to the differentiating neural stem cells exogenously overexpressing CYFIP1, the effect of norathyriol on excessive dendritic arborization generated by CYFIP1 overexpression was investigated. By immunofluorescent staining method using an antibody against pan-neurofilament and an antibody against PSD (post-synaptic density)-95, which was expressed in the post-synaptic dendrite of synapses, the neurite length and dendritic spine number were measured and quantified. In addition, by DAPI staining, the nuclei of the neural cells were visualized.

### 6-1. Isolation and Culture of Hippocampus-Derived Neural Progenitor Cells

Hippocampus-derived neural progenitor cells were isolated from the forebrain of E16 mouse embryos. SD-rat gestational age 16-day mice (Orient Bio) were purchased and the mice were anesthetized with CO₂ deeply, and then the embryos were taken out. The embryos were immersed in Ca²⁺/Mg²⁺-free HBSS (Invitrogen) and the forebrain was isolated using a sterile injection needle under a dissecting microscope. While changing HBSS, the forebrain was separated into the left and right hemispheres using a forcep, and meninges were peeled off, and then the hippocampal eminence was separated. After washing the separated and collected tissue with cold N2 culture medium twice, 37° C. N2 culture medium was added and cells were slowly and mechanically isolated using a pipette. As the pipette, a Pasteur pipette coated with 1% BSA was used after making the pipette narrow and soft in diameter by firing the tip. After waiting for 3 minutes to allow undissolved tissue or vascular cells to settle, the supernatant was carefully taken as dissolved cells. These tissues were collected and mechanically isolated with the pipette in Ca²⁺/Mg²⁺-free HBSS (Invitrogen) buffer. The cells were seeded at 19,000 cells/cm² in a 35 mm culture dish coated with 15 μg/ml poly-L-ornithine and 1 μg/ml fibronectin (Sigma), and cultured using a serum-free N2 medium (medium for proliferation) in which 10 ng/mL bFGF (Invitrogen) was added under a condition of 5% CO₂. The culture medium was changed after 6 hours of primary culture, and at the same time the next day, the culture medium was additionally changed. 90% of the medium for proliferation was changed every 2 days. After 2 days, when the cells were proliferated twice or more, subculture was conducted.

After culturing the neural stem cells in the medium for proliferation for 3 days, the cells were detached using 0.05% trypsin/EDTA, and subculture was carried out once to twice. The neural progenitor cells were seeded to a 12 mm glass cover glass (Velco) for immunostaining and to a 100 mm culture dish for immunoblot analysis, and then they were cultured in an N2 medium for proliferation containing bFGF for 1 day. After that, they were cultured in a medium comprising FGF continuously for 2-3 days under a proliferation condition, and they were cultured in a medium for differentiation comprising no FGF for 3-7 days under a differentiation condition, and norathyriol was treated at 10, 50, and 100 nM, respectively. In the control group (vehicle), physiological saline solution or DMSO in which the compound was dissolved was added at the same concentration in the same volume.

6-2. Introduction of CYFIP1 Expression Vector into Hippocampal Neural Progenitor Cells by Electroporation

After 3 days of culturing primary neural progenitor cells, the cells were isolated from a culture dish with 0.05% trypsin, and CYFIP1 plasmid DNA (pDEST-CYFIP1-GFP) was added to the cells of 2×10⁶ at a 1.5 μg DNA ratio, and after mixing neucleofector solution (Lonza, S-06387) 82 μl and Supplement1 (Lonza, S-06372) 18 μl contained in Amaxa Rat Neuronal Stem Cell Nucleofector Kit (Lonza, VPG-1005), they were introduced using 4D-Nucleofector X Unit (Lonza, AAF-1001X) electroporator. The introduced cells were seeded on a cover glass in a 24 well, and cultured in an N2 (+bFGF) medium for proliferation for 24 hours, and then grown in an N2 (−FGF) medium for differentiation, and the medium was changed by 50% every 2 days. A drug was treated in a 2-day cycle from 4 days after differentiation for 4 days. After staining, the effect was investigated by scanning with a confocal laser microscope (Zeiss, LSM800). In case of confirmation of the number of cells and data analysis, the number of cell nuclei labeled with DAPI was counted and the cells expressing green fluorescence overlapped with DAPI staining was counted as the CYFIP1-GFP expressing cells. The staining intensity was measured using a confocal laser microscope program. Non-specific signals appearing at the edges of the slide were not included. For data analysis, the control group and experimental group were compared by One-way analysis of variance (Anova). The statistical significance of the data was set at p<0.05.

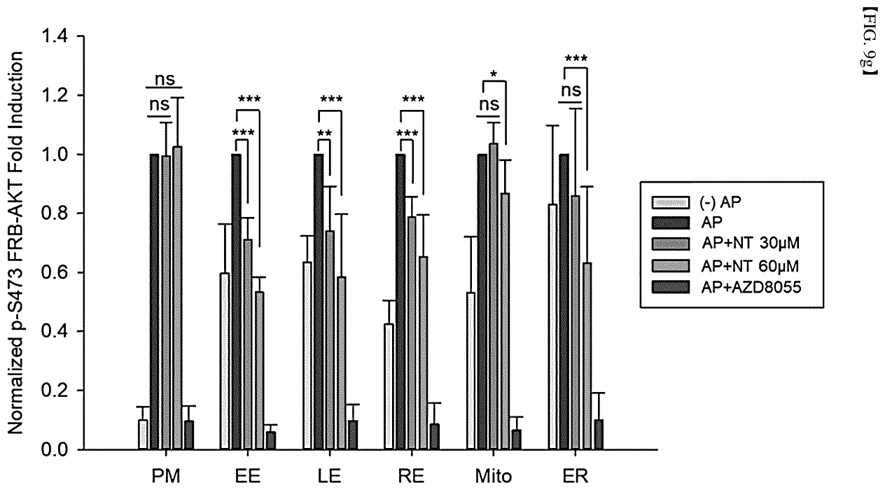

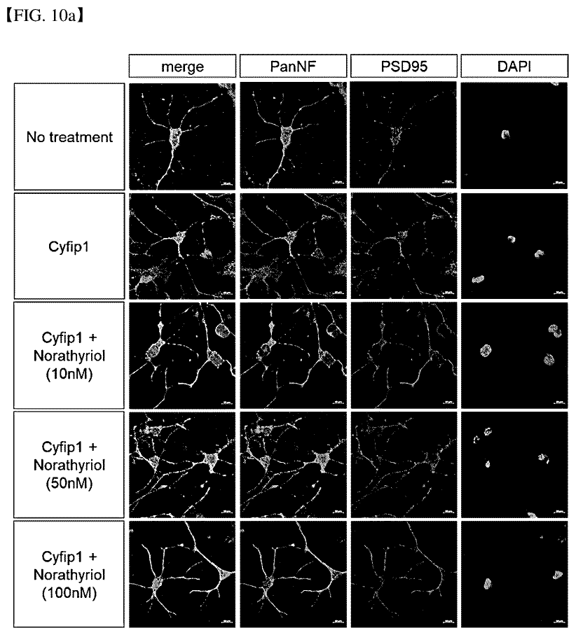

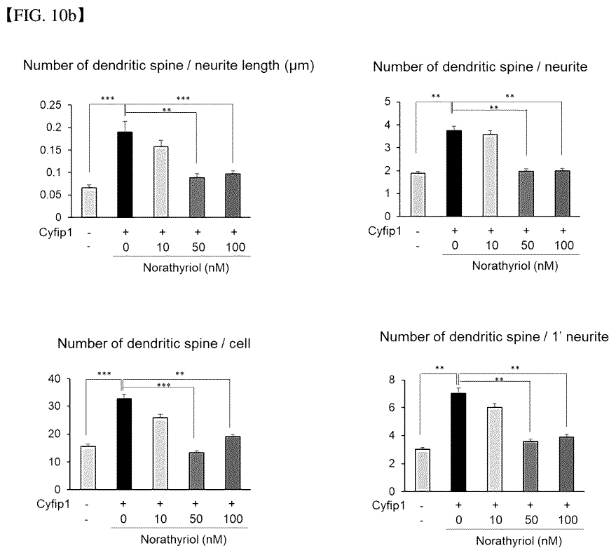

As a result, as shown in FIGS. 10a and 10b, the number of dendritic spines per unit neurite length in the CYFIP1-oeverexpressing neurons increased by about 2.5 times compared to the control group in which CYFIP1 was not introduced. When norathyriol was treated to the CYFIP1-oeverexpressing neurons, the number of dendritic spines per unit neurite length was reduced in a concentration-dependent manner. A decrease in the number of dendritic spines was observed from the 10 nM concentration of norathyriol, and it was reduced at a statistically significant level by 50% or less on average at 50 nM and 100 nM. In addition, the number of dendritic spines per cell and the number of dendritic spines in primary branching neurites were also reduced by norathyriol in a similar tendency.

From such experimental results, it was confirmed that norathyriol inhibited excessive dendritic arborization phenomena caused by CYFIP1 overexpression.

### Experimental Example 7. Evaluation of mGluR-Dependent LTD Controlling Efficacy of Norathyriol and Athyriol in Primary Cultured Neurons

The present experiment was performed to evaluate the effects of norathyriol and athyriol on mGluR-dependent LTD dysregulation, which was considered as a primary cause of autism spectrum disorder symptoms, in particular, specifically, effects on 1) mGluR-dependent mTORC2 activation, 2) mGluR-dependent Arc expression and 3) mGluR-dependent LTD occurrence.

7-1. Confirmation of Results of Evaluation of Inhibitory Efficacy of Norathyriol on mGluR-Dependent mTORC2 Activation in Dendritic Spines of Primary Cultured Cerebral Neurons

By combining Loca-TOR2 assay and immunofluorescence staining, changes in mTORC2 activity located in the late endosome during the induction process of long term depression (LTD) occurring in dendritic spines of primarily cultured cerebral neurons were investigated, and the inhibitory effect of norathyriol on these changes was investigated.

Specifically, mCherry-FRB-AKT2 and Rab7-FKBP were introduced using a lentiviral vector on the primarily cultured cerebral neuron on the DIV5 day, and AP21967 was treated on the DIV17 day to induce binding of FRB and FKBP, and thereby, mCherry-FRB-AKT2 was located in the late endosome. After that, DHPG, which is an mGluR agonist inducing long-term depression, was treated. As the mCherry-FRB-AKT2 was recruited to the late endosome by AP21967, AKT Ser473 may be phosphorylated by mTORC2 located in the late endosome, and the degree of Ser473 phosphorylation of mCherry-FRB-AKT2 becomes an indicator exhibiting mTORC2 activity located in the late endosome. In addition, by measuring Ser473 phosphorylation staining of mCherry-FRB-AKT2 overlapped with PSD-95 staining, which is a protein located in dendritic spines, by immunofluorescent staining, changes in mTORC2 activity located in the late endosome in the dendritic spines can be investigated. After staining using a phospho-Ser473 AKT antibody by immunofluorescent staining, the degree of AKT Ser473 phosphorylation was quantified by counting the number of each fluorescent puncta larger than 0.4 μm or more in size in the dendrites (green). In addition, the dendritic spines were observed by staining with PSD95 antibody (yellow), and the location of mCherry-FRB-AKT2 was investigated by observing mCherry fluorescence (cherry color).

### 7-1-1. Isolation and Culture of Cerebral Neurons

SD-rat gestational age 16-day mice (Orient Bio) were purchased and the mice were anesthetized with CO₂ and then embryos were taken out. Until the forebrain was isolated and the meninges were removed, it was performed by the same method as the hippocampal culture, and when the cerebrum was isolated, the dorsal cortex or ventral cortex was cut with a bended syringe needle, and it was collected in new HBSS. The tissue isolated and collected was transferred to a 15 ml tube with a Pasteur pipette, and then in order to remove the remaining meninges and blood vessels, and the like, 2 ml HBSS and 0.25% trypsin of 2 ml were added, and it was reacted in a 37° C. shaker for 10-15 minutes. The cells were settled and the supernatant was removed leaving 1-2 ml, and then trypsin was inactivated by adding DMEM comprising the equal amount of 20% FBS. After centrifugation at 700 rpm for 15 minutes, the supernatant was discarded all and the collected tissue was released, and then Neurobasal culture medium (+glutamate) of 2 ml was added, and titrated with a pipette, and the cells were isolated by changing to a pipette with a narrow end. The cells were filtered in a 40 μm nylon mesh-cell strainer (Falcon #2340) inserted into a 50 ml tube, and the strainer was washed using NB culture medium of 10˜20 ml. The number of cells was measured with a hemocytometer, and then the cells were seeded in a coated culture dish. The cells of 5×10⁵ were seeded in a 100 mm culture dish pre-coated with 1 mg/ml Poly-D-Lysine (sigma, P0899, 50 mg) and Laminin (Invitrogen, 23017-015, 1 mg), and cultured using Neurobasal complete culture medium (+glutamax, B27, Pen/stryp) under a condition of 5% CO₂. The culture medium was changed after 6 hours of primary culture, and the culture medium was additionally changed at the same time the next day. After that, the culture medium was exchanged by 50% in a 2-day cycle and they were differentiated for 18 days.

### 7-1-2. LocaTOR2-Immunofluorescent Staining

FRB-AKT2 lentivirus and Rab7-FKBP lentivirus were transduced into the differentiated cerebral neurons to express FRB-AKT and Rab7-FKBP, and then AP21967 was treated for 40 minutes, and then they were fixed with PBS comprising 4% PFA for 15 minutes. After washing with PBS twice and PBST once, permeabilization was performed using 0.5% Triton X-100-PBST for 10 minutes. The cells were blocked with 2% BSA-PBST or 5% normal serum (Normal donkey serum: Jackson lab, 017-000-121, Normal horse serum: Sigma, H0146), and primary antibodies were treated in 2% BSA-PBS, and reacted at 4° C. overnight. On the next day, after washing with PBST, secondary antibodies were treated in PBST and reacted at a room temperature for 1 hour. After staining cell nuclei with DAPI (1 μg/mL, Sigma), they were mounted in a slide glass and observed with a confocal laser microscope. For the method, Heo et al. (Neurosci. Lett. (2009) 450:45-50) and Han et al. (J. Med. Food (2012) 15:413-417) were referred. panAkt (Cell signaling, #4691, 1:1000), pAkt-S473 (Cell signaling, #4060, 1:400), PSD95 (Invitrogen #MA1-046, 1:500), and Arc (Santa Cruz Biotech, sc-17839, 1:500) for the used primary antibodies, and Alexa 488 (Invitrogen, #A21202, 1:700), Cy3 (Jackson lab, #715-165-151, 1:500), and Alexa 488 (Jackson lab, #711-546-152, 1:700), and the like for the secondary antibodies were used.

### 7-1-3. LTD Measurement

A brain extracted from a normal mouse was cut with vibraome, and horizontal hippocampal brain slices with a thickness of 300 μm were prepared. The hippocampal brain slices were perfused with aCSF at 32° C. for 1 hour at minimum, and then flowed to aCSF comprising DMSO or 0.5 μM athyriol at 32° C. for 2 hours. After exciting the Schaffer collateral and commissural fibers with a bipolar stimulating electrode located in CA1 stratum radiatum, field excitatory postsynaptic potentials (fEPSPs) were measured with a recording electrode located in stratum radiatum. After establishing a baseline of stable fEPSP for the first 10 minutes, 100 μM DHPG was treated for 10 minutes. After that, the recovery of the inhibited fEPSP was measured while washing with aCSF.

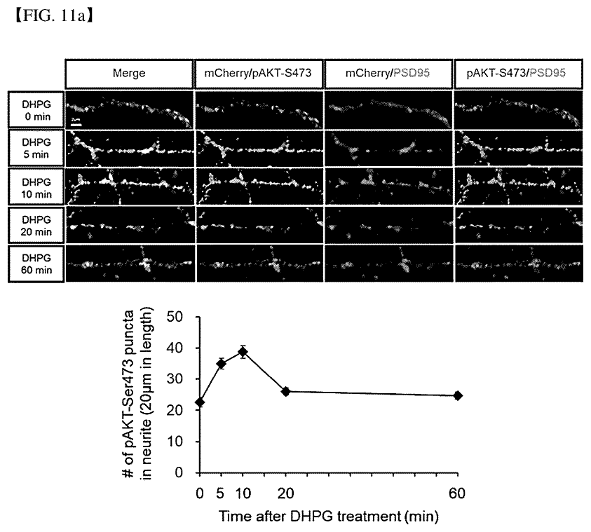

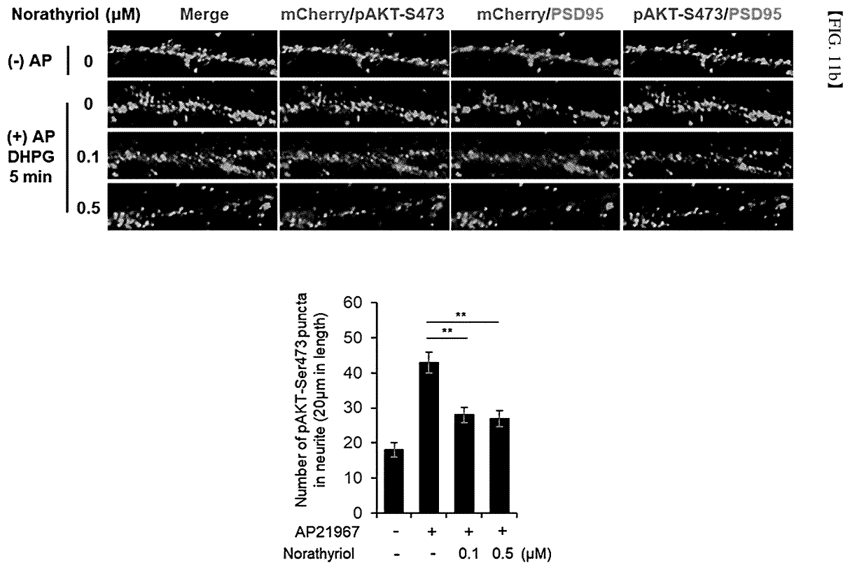

As a result, as shown in FIG. 11a, after 5 minutes and 10 minutes of treating DHPG, Ser473 phosphorylation of mCherry-FRB-AKT2 located in the late endosome of dendritic spines increased by 55% and 72%, respectively, and after 20 minutes, it was reduced to the level of the control group. mCherry+PSD95 shows that mCherry-FRB-AKT2 localized at the inside of dendritic spines, and pAKT+PSD95 shows that mCherry-FRB-AKT2 located in the late endosome of dendritic spines was phosphorylated at Ser473 by DHPG treatment. Therefore, by the number of the dendritic spines stained with mCherry+pAKT+PSD95, it was confirmed that the mTORC2 activity located in the late endosome of the dendritic spines increased by DHPG treatment.

In addition, as shown in FIG. 11b, when the primarily cultured cerebral neurons were differentiated for DIV17 days, and DHPG, which is an mGluR agonist inducing long-term depression, was treated, phosphorylation of AKT=S473, which is an mTORC2 substrate induced to the late endosome in dendritic spines (PSD95-yellow staining), significantly increased after 5 minutes and 10 minutes of treating DHPG (green staining). On the other hand, in the neurons in which norathyriol was pretreated, AKT-Ser473 phosphorylation induced by DHPG was measured similarly to the control group level. From this experimental result, it was confirmed that mTORC2 located in the late endosome of dendritic spines was activated by mGluR activation in synapses of neurons, and this mGluR-dependent mTORC2 activation was inhibited by norathyriol.

### 7-2. Evaluation of Inhibitory Efficacy of Norathyriol on mGluR-Dependent Arc Protein Expression in Synapses of Cerebral Neurons

When mGluR is activated in dendritic spines of cerebral neurons, Arc protein is synthesized, and the Arc protein promotes endocytosis of an AMPA receptor to form LTD. In addition, it was found that mTORC2, not mTORC1, mediates mGluR-dependent LTD formation. Since it was found that mGluR-dependent mTORC2 activation was inhibited by norathyriol in dendritic spines of cerebral neurons in the above experimental example, as the following experiment, the inhibitory efficacy of norathyriol on mGluR-dependent Arc protein expression was evaluated.

Specifically, DHPG was treated to cerebral neurons, which were obtained by primary culture of white mouse embryo E16 cerebral regions and differentiated for 18 days (DIV21), for 5 minutes or 10 minutes to activate mGluR. As an Arc antibody, Arc protein (green) and PSD95 (red), which is a marker antibody of postsynaptic density of a synapse, were under

After double immunofluorescent staining of Arc protein with an Arc antidody (green) and PSD95, a marker of postsynaptic density of synapse, with an PSD95 antibody (red), Arc expression in dendritic spines was quantified as the number of Arc+PSD95 double stained puncta per 20 μm neurite length by confocal scanning microscopic scanning.

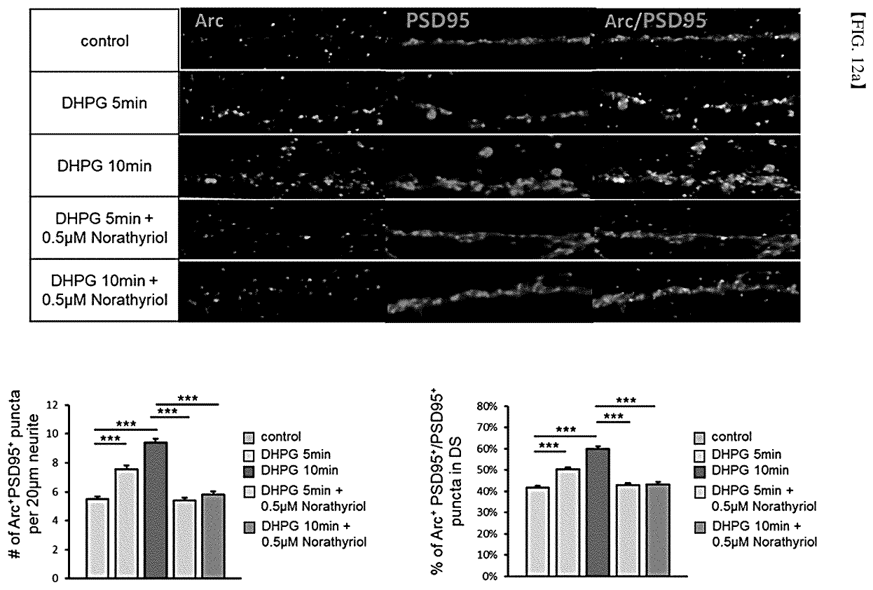

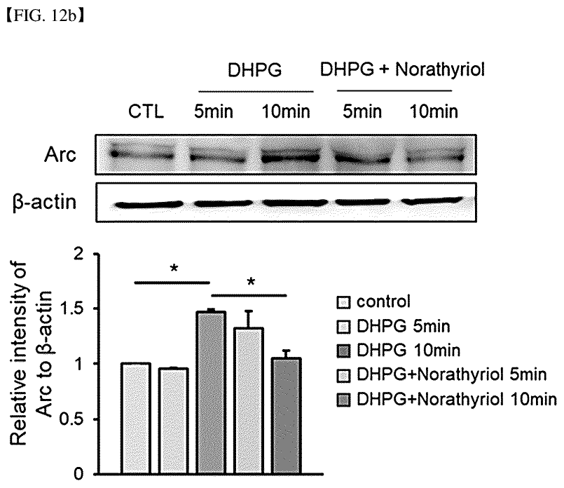

As a result, as shown in FIG. 12a, Arc synthesis in the dendritic spines increased up to 10 minutes after treating DHPG. However, in the cerebral neurons pretreated with norathyriol for 2 hours before treating DHPG, the increase of Arc synthesis by DHPG was not found. In addition, as shown in FIG. 12b, an immunoblot analysis of cell lysate prepared from the cerebral neurons confirmed that the increase of Arc protein synthesis was inhibited in a dose-dependent manner by norathyriol. From this experimental result, it was confirmed that mGluR-dependent Arc protein synthesis in dendritic spines of neurons was inhibited by norathyriol.

7-3. Evaluation of Inhibitory Efficacy of Athyriol on mGluR-Dependent LTD Formation in Hippocampal Brain Slices

Arc protein synthesized as mGluR is activated promotes AMPA receptor endocytosis, thereby inducing LTD formation. Because it was confirmed that mGluR-dependent Arc protein synthesis was inhibited by norathyriol in the above experimental example, the inhibitory efficacy of athyriol, which had a better mTORC2 inhibitory ability and delivery ability into neurons in brain slices than norathyriol, on mGluR-dependent LTD in the hippocampal brain slices obtained from normal mice was evaluated.

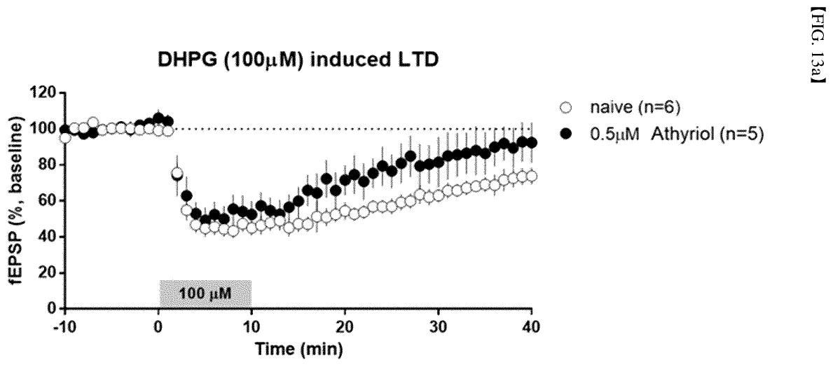

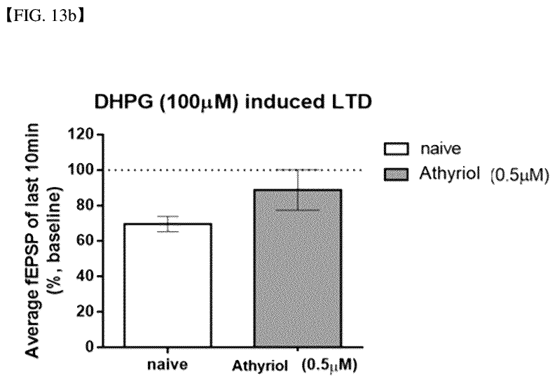

FIG. 13a, and FIG. 13b, which are results thereof, show graphs of comparing and quantifying a change in fEPSP over time, and the value of average fEPSP for the last 10 minutes normalized to the 10-minutes base line. In the control group in which DMSO was treated as a vehicle, fEPSP stayed at a level of 70˜80% up to 40 minutes after treating DHPG, so it could be confirmed that LTD occurred. In contrast thereto, fEPSP was recovered by 90% after 40 minutes of treating DHPG in the hippocampal brain slices pretreated with athyriol at a concentration of 0.5 μM for 2 hours. Accordingly, it could be confirmed that athyriol inhibited mGluR-dependent LTD occurring in the hippocampus.

### Experimental Example 8. Evaluation of Efficacy of Memory Improvement and Sociability Enhancement of Athyriol Administration in Normal Mice

In the above Experimental example 1, it was confirmed that Pten KO mice administered with athyriol at a dose of 10 mg/kg showed the better memory ability and sociability than the normal control group. From this result, the possibility that athyriol improves the memory ability and sociability in normal mice was suggested. In order to verify this possibility, the efficacy of athyriol on memory ability and sociability of normal animals was investigated. A vehicle (30% DMSO) or athyriol was intraperitoneally injected at a dose of 5 mg/kg into normal mice (n=10), and from the 7th day after administration, the memory ability was evaluated by the same method as the Y-type maze test of Experimental example 1-2, and the sociability was evaluated by the same method as the 3 chamber test of Experimental example 1-6.

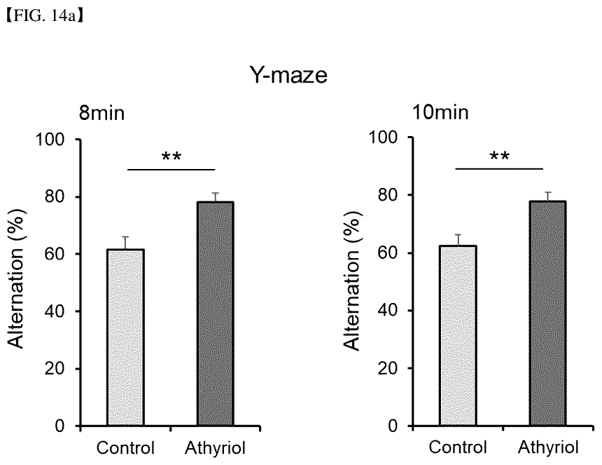

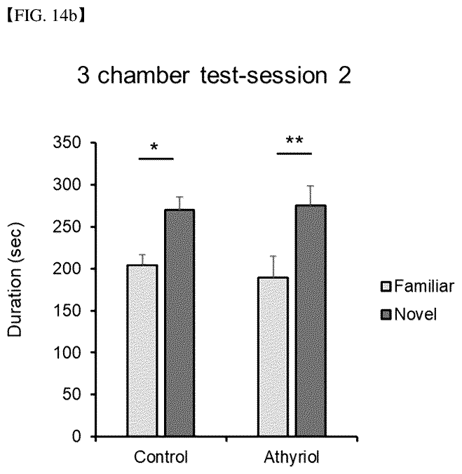

As a result, as shown in FIG. 14a, the mice administered with athyriol at a dose of 5 mg/kg showed the improved memory ability by 25% or more compared to the control group in which only DMSO was administered as a vehicle in the Y-type maze test which examined the memory ability. In addition, as shown in FIG. 14b, in the sociability measurement-2 session of the 3 chamber test examining sociability, the mice administered with athyriol at a dose of 5 mg/kg showed the increase by 11% in the ratio of searching time for familiar mice to searching time for unfamiliar mice compared to the control group in which only DMSO was administered. From this experimental result, it was confirmed that athyriol administration improved the memory ability of normal mice and enhanced their sociability.

### [Preparation Examples of Drugs]

The active substance according to the present invention can be formulated in various forms depending on the purpose. The followings are examples of some formulations containing the active substance according to the present invention as an active ingredient, and the present invention is not limited thereto.

<Drug preparation example 1> Preparation of a powder

| | | |
|---|---|---|
| Active substance | 2 | g |
| Lactose | 1 | g 1 |

After mixing the above components, a powder was prepared by filling them in an air tight bag.

<Drug preparation example 2> Preparation of a tablet

| | | |
|---|---|---|
| Active substance | 100 | mg |
| Corn starch | 100 | mg |
| Lactose | 100 | mg |
| Magnesium stearate | 2 | mg |

After mixing the above components, according to a common preparation method of a tablet, a tablet was prepared by tableting.

<Drug preparation example 3> Preparation of a capsule

| | | |
|---|---|---|
| Active substance | 100 | mg |
| Corn starch | 100 | mg |
| Lactose | 100 | mg |
| Magnesium stearate | 2 | mg |

After mixing the above components, according to a common preparation method of a capsule, they were filled in a gelatin capsule to prepare a capsule.

<Drug preparation example 4> Preparation of an injection

| | |
|---|---|
| Active substance | 10 μg/m [symbol] |
| Dilute hydrochloric acid BP | Until pH 3.5 |
| Sodium chloride BP for injection | 1 m [symbol] at maximum |

The active substance according to the present invention was dissolved in sodium chloride BP for injection at an appropriate volume, and the pH of the produced solution was adjusted to pH 3.5 using dilute hydrochloric acid BP, and the volume was adjusted using sodium chloride BP for injection, and they were sufficiently mixed. The solution was filled in 5 ml type I ample made of transparent glass, and was encapsulated under an upper grid of air by dissolving glass, and sterilized by autoclaving at 120° C. for 15 minutes or more to prepare an injection solution.

<Drug preparation example 5> Preparation of a nasal spray

| | | |
|---|---|---|
| Active substance | 1.0 | g |
| Sodium acetate | 0.3 | g |
| Methyl paraben | 0.1 | g |
| Propyl paraben | 0.02 | g |

| | |
|---|---|
| Sodium chloride | Suitable amount |
| HCl or NaOH | pH adjustment suitable amount |
| Purified water | Suitable amount |

According to a conventional preparation method of a nasal spray, it was prepared so that 3 mg of active substance is comprised per 1 mL of salt water (0.9% NaCl, w/v, a solvent was purified water), and this was filled in an opaque spray container and sterilized to prepare a nasal spray.

<Drug preparation example 6> Preparation of a liquid

| | | |
|---|---|---|
| Active substance | 100 | mg |
| Isomerized glucose syrup | 10 | g |
| Mannitol | 5 | g |

| | |
|---|---|
| Purified water | Suitable amount |

According to a conventional preparation method of a drug, each component was added to purified water and was dissolved, and lemon flavor was added, and then the above components were mixed, and then purified water was added to adjust the total volume to 100 mL, and then it was filled in a brown bottle and sterilized to prepare a liquid.

So far, the present invention has been examined in priority of preferable examples thereof. Those skilled in the art to which the present invention pertains will understand that the present invention can be implemented in a modified form without departing from essential characteristics of the present invention. Therefore, the disclosed examples should be considered from an illustrative point of view rather than a limitative point of view. The scope of the present invention is particularly indicated in claims rather than the aforementioned description, and it should be construed that all differences within the equivalent range thereto are included in the present invention.

1. A method for prevention or treatment of mTORopathy comprising administering a pharmaceutical composition comprising athyriol or a pharmaceutically acceptable salt thereof as an active ingredient.

2. The method according to claim 1, wherein the mTORopathy is any one or more selected from the group consisting of epilepsy, autism spectrum disorder (ASD), macrocephaly, tuberous sclerosis complex (TSC), seizure, fragile X syndrome, PTEN harmartoma tumor syndrome (PHTS), neurofibromatosis and intellectual disability.

3. The method according to claim 2, wherein the autism spectrum disorder is any one or more selected from the group consisting of autism, Asperger's disorder, Pervasive Development Disorder-Not Otherwise Specified (PDD-NOS), Rett's disorder, Childhood Disintegrative Disorder and Autism Spectrum Disorder.

4. The method according to claim 1, wherein the composition selectively inhibits mTORC2 activity.

5. The method according to claim 4, wherein the composition inhibits mTORC2 activity twice or more than mTORC1 activity.

6. The method according to claim 4, wherein the composition selectively inhibits activity of mTORC2 located in endosome.

7. The method according to claim 1, wherein the composition further comprises any one or more selected from the group consisting of norathyriol, mangiferin, neomangiferin and pharmaceutically acceptable salts thereof.

8. The method according to claim 1, wherein the composition enhances memory ability.

9. The method according to claim 1, wherein the composition improves anxiety disorder.

10. A method for prevention or improvement of mTORopathy comprising administering a food composition comprising athyriol or a sitologically acceptable salt thereof as an active ingredient.

11. The method according to claim 10, wherein the mTORopathy is any one or more selected from the group consisting of epilepsy, autism spectrum disorder (ASD), macrocephaly, tuberous sclerosis complex (TSC), seizure, fragile X syndrome, PTEN harmartoma tumor syndrome (PHTS), neurofibromatosis and intellectual disability.

12. The method according to claim 11, wherein the autism spectrum disorder is any one or more selected from the group consisting of autism, Asperger's disorder, Pervasive Development Disorder-Not Otherwise Specified (PDD-NOS), Rett's disorder, Childhood Disintegrative Disorder and Autism Spectrum Disorder.

13. The method according to claim 10, wherein the composition selectively inhibits mTORC2 activity.

14. The method according to claim 13, wherein the composition inhibits mTORC2 activity twice or more than mTORC1 activity.

15. The method according to claim 13, wherein the composition selectively inhibits activity of mTORC2 located in endosome.

16. The method according to claim 10, wherein the composition further comprises any one or more selected from the group consisting of norathyriol, mangiferin, neomangiferin and sitologically acceptable salts thereof.

17. A method for enhancing memory, comprising administering a food composition comprising athyriol or a sitologically acceptable salt thereof as an active ingredient.

18. A method for enhancing anxiety disorder, comprising administering a food composition comprising athyriol or a sitologically acceptable salt thereof as an active ingredient.
